(19) United States
(12) Patent Application Publication
(10) Pub. No.: US 2023/0064975 A1
(43) Pub. Date: Mar. 2, 2023

(54) COMPOSITION AND METHOD FOR AN ANTIBIOTIC-INDUCING IMBALANCE IN MICROBIOTA

(71) Applicants: PSOMAGEN, INC., Rockville, MD (US); MACROGEN INC., Seoul (KR)

(72) Inventors: Zachary Apte, San Francisco, CA (US); Jessica Richman, San Francisco, CA (US); Daniel Almonacid, San Francisco, CA (US); Jeongsun Seo, Seoul (KR); Inseon KIM, Bucheon-si (KR); Dongjun Kim, Seoul (KR); Nak Jung Kwon, Seoul (KR); Su Hong, Rockville (MD)

(21) Appl. No.: 17/961,863

(22) Filed: Oct. 7, 2022

Related U.S. Application Data
(63) Continuation of application No. PCT/KR2021/004438, filed on Apr. 8, 2021.
(60) Provisional application No. 63/006,757, filed on Apr. 8, 2020.

(51) Int. Cl. A61K 35/742; A61K 35/747; A61K 35/745; C12Q 1/04; C12Q 1/689

(52) U.S. Cl. CPC A61K 35/742; A61K 35/747; A61K 35/745; C12Q 1/04; C12Q 1/689

(57) ABSTRACT

A composition and a method for an antibiotic-inducing imbalance in microbiota, or specifically, a microbiota recovery composition for an antibiotic-inducing imbalance of gut microbiota are provided.

### CROSS-REFERENCE

This application claims the priority benefit of: U.S. Ser. No. 63/006,757 entitled “selection of candidate bacteria for use as live biotherapeutics (LBP) for antibiotic recovery treatment” filed on Apr. 8, 2020.

### TECHNICAL FIELD

The present invention relates to a composition and method for an antibiotic-inducing imbalance in microbiota, or specifically, a microbiota recovery composition for an antibiotic-inducing imbalance of gut microbiota, a method of selecting a microorganism useful for recovering an antibiotic-inducing imbalance of gut microbiota in a subject, and a use of amelioration or treatment of an antibiotic-inducing imbalance of gut microbiota in a subject.

### BACKGROUND ART

Antibiotic consumption has strong effects on the gut microbiota through direct or indirect mechanisms. In particular, some bacterial taxa are severely affected, and in some cases, they disappear from the community and are not easily recovered. Extensive use of antibiotics also negatively impacts human health.

A growing number of studies have shown that antibiotics can result in microbial dysbiosis, and the disruption of gut microbiota in neonates and adults contributes to numerous diseases, including diabetes, obesity, inflammatory bowel disease, asthma, rheumatoid arthritis, depression, autism, and superinfection in critically ill patients.

It is unlikely that the same species colonize the gut in the same way than before the exposure. Reports have shown that, even though Lactobacillus spp. can recover, many other important taxa do not recover after 6 months (and/or suitable time) of antibiotics exposure. These include some members of Bifidobacterium genus which contribute to protection against pathogens and activation of immune response, some butyrate and propionate producers from Coprococcus and Eubacterium genus and also, some species associated to polysaccharides digestion. Poor abundances of those species can trigger decreased immunity and some other undesired effects such as diarrhea.

At the same time, depletion of those species may lead to the blooming of opportunistic bacteria, such as some members of Clostridium spp. (e.g., especially C. difficile), whose ability to survive relies on the spores production.

Many of bacterial taxa have specific functions, which are critical for the general health of the host. It would be desirable to identify these organisms, and be able to recolonize the gut microbiota with them after a course of antibiotics.

### DISCLOSURE

### Technical Problem

An embodiment of the present invention is to provide a microbiota recovery composition for an antibiotic-inducing imbalance of gut microbiota.

Another embodiment is to provide a method of selecting a microorganism useful for recovering an antibiotic-inducing imbalance of gut microbiota in a subject.

A further embodiment is to provide a method of ameliorating or treating an antibiotic-inducing imbalance of gut microbiota in a subject providing, or administering a microbiota recovery composition for an antibiotic-inducing imbalance of gut microbiota, to a subject with the antibiotic-inducing imbalance of gut microbiota.

A still further embodiment is to provide a use of amelioration or treatment of an antibiotic-inducing imbalance of gut microbiota in a subject, or a microbiota recovery composition, in the use of amelioration or treatment of an antibiotic-inducing imbalance of gut microbiota in a subject.

### Technical Solution

The following description of the embodiments is not intended to limit the embodiments, but rather to enable any person skilled in the art to make and use.

Embodiments can include identification of, approaches associated with, suitable therapeutic compositions (e.g., live biotherapeutic compositions) including and/or any suitable method processes and/or system components including and/or associated with one or more species (and/or any suitable approaches described herein can be used for identifying any suitable microorganisms from any suitable type of taxa level; etc.) that are currently included in probiotics (and/or suitable consumables and/or therapeutics and/or therapeutic compositions) that are depleted (e.g., decrease in composition amount; lost; reduced; after antibiotics usage (and/or during), and/or that we can include in a therapeutic composition (e.g., new blend, etc.) of LBPs (and/or suitable consumables (e.g., live biotherapeutics, probiotics, prebiotics, etc.) and/or therapeutics).

Additionally or alternatively, embodiments can include identifying new short-chain fatty acids (SCFA)-producer species (and/or suitable microorganism taxa) that are not included in any previous probiotic (and/or suitable consumables; and/or therapeutics etc.). Any suitable taxa described herein (and/or identifiable by approaches described herein) can be used in one or more LBPs (and/or suitable consumables (e.g., live biotherapeutics, probiotics, prebiotics, etc.) and/or therapeutics.

In examples, an objective that can be achieved includes identifying bacteria that show a decrease after antibiotics consumption, such as candidates for LBPs and/or suitable consumables (e.g., live biotherapeutics, probiotics, prebiotics, etc.) and/or therapeutics.

Embodiments of a method can include Identifying bacteria that are depleted and/or otherwise affected after (and/or during) antibiotic consumption and/or whose functions are relevant to preserve health condition.

Embodiments can include therapeutic compositions, processes associated with, determination of, generation of, and/or can otherwise be associated with any suitable combinations of microorganism taxa (e.g., bacterial taxa, etc.) that can be included in a probiotic formulation (and/or suitable consumable and/or therapeutic composition; etc.), such as for gut microbiota (and/or suitable body site microbiome) recovery during and/or after antibiotics exposure.

Embodiments can include identifying bacteria and/or suitable microorganism taxa to be used to recolonize the gut and/or suitable body sites, during and/or after antibiotics treatment, such as to be included in a LBP formulation, such as with the goal of recovering relevant functions such as: pathogen inhibition, degradation of polysaccharides, degradation of mucin, short-chain fatty acids production, production of conjugated linoleic acid, production of enterolactone, production of gamma aminobutyric acid (GABA), production of indole, and/or suitable microorganism-related functionality.

More specifically, the disclosure provides a microbiota recovery composition for an antibiotic-inducing imbalance of gut microbiota comprising at least a bacterium which is decreased relative abundance or depleted during and/or after the antibiotic consumption or antibiotic exposure, or the negatively affected functions which are relevant to preserve health condition. The functions are one or more of the described can include and/or be associated with all, or some of the following properties: pathogen inhibition, degradation of polysaccharides, degradation of mucin, short-chain fatty acids production, production of conjugated linoleic acid, production of enterolactone, production of GABA, production of indole, and/or suitable microorganism-related functionality.

The microbiota recovery composition can recolonize the gut and/or suitable body sites, or recover relevant functions such as at least one selected from the group consisting of pathogen inhibition, degradation of polysaccharides, degradation of mucin, short-chain fatty acids production, production of conjugated linoleic acid, production of enterolactone, production of gamma aminobutyric acid (GABA), production of indole, and suitable microorganism-during and/or after antibiotics exposure, or preferably, pathogenesis and/or short-chain fatty acids production.

In addition, at least bacterium to be included in the microbiota recovery composition can be extracted or excluded based on the functional features of bacterium, which can be for example at least one selected from the group consisting of pathogenesis, pathogen inhibition, degradation of polysaccharides, degradation of mucin, short-chain fatty acids production, production of conjugated linoleic acid, production of enterolactone, production of GABA, and production of indole, or preferably at least one feature selected from the pathogenesis and the short-chain fatty acids production. Particularly, candidate bacteria can be extracted based on the short-chain fatty acids production or excluded based on the pathogenesis from the microbiota recovery composition. One of the most important functions that are usually lost after antibiotics treatment, as described according our statistical analyses, is the production of short-chain fatty acids (SCFA), such as propionate or butyrate. This important function helps to prevent severe diarrhea after the antibiotic usage, among other anti-inflammatory features.

The present inventors have identified the bacterial species being currently available by testing whether they are decreased or depleted after antibiotics usage, and determine them as components of the microbiota recovery composition. Particularly, Bifidobacterium adolescentis, Bfidobacterium animalis, Bifidobacterium breve, Bifidobacterium catenulatum, Bifidobacterium pseudocatenulatum, Bindobacterium stercoris, Bacteroides xylanisolvens, Lactobacillus rhamnosus and Lactococcus lactis described in Table 4 are included in probiotics, and are identified as agents for recovering the antibiotic-inducing imbalance in the present invention.

Also, the present inventors have identified some new SCFA-producing species that are not used as a component of probiotics before. Faecalibacterium prausnitzii, Roseburia faecis, Roseburia hominis, Roseburia intestinalis, Anaerostipes caccae, Aneerostipes rhamnosivorans, Eubacterium limosum, Eubacterium sp. ARC.2, Subdoligranulum variabile, Akkermansia muciniphila, Bifidobacterium crudilacds, Bifidobacterium dentium, Bifidobacterium thermacidophilum, Methanobrevibacter smithii, Roseburia sp. 499, Bacteroides dorei, Bacteroides massiliensis, Bacteroides plebeius, Bacteroides sp. 35AE37, and Bacteroides thetaiotaomicron described in Table 4 have not been known as a component of probiotic, and are firstly identified as an agent for recovering the antibiotic-inducing imbalance in the present invention.

In a specific embodiment, a microbiota recovery composition for an antibiotic-inducing imbalance of gut microbiota comprises at least a bacterium selected from the group consisting of Faecalibacterium prausnitzii, Roseburia faecis, Roseburia hominis, Roseburia intestinalis, Anaerostipes caccae, and Anaerostipes rhamnosivorans. The combination of all of them, or a subset of them, can be used for treatment, diagnostics, and/or any suitable purpose. One or more of the described can include and/or be associated with all, or some of the following properties: pathogen inhibition, degradation of polysaccharides, degradation of mucin, short-chain fatty acids production, production of conjugated linoleic acid, production of enterolactone, production of GABA, production of indole, and/or suitable microorganism-related functionality.

Preferably, the microbiota recovery composition comprises at least a bacterium selected from the group consisting of Faecalibacterium prausnitzii, Roseburia faecis, Roseburia hominis, Roseburia intestinalis, Anaerostipes caccae, Anaerostipes rhamnosivorans, Eubacterium limosum, Subdoligranulum variabile, Lactobacillus rhamnosus, Akkermansia muciniphila, Bifidobacterium adolescentis, Bifidobacterium catenulatum, Bifidobacterium pseudocatenulatum, and Bifidobacterium stercoris. Alternatively, the microbiota recovery composition comprises at least a bacterium selected from the group consisting of Faecalibacterium prausnitzii, Roseburia faecis, Roseburia hominis, Roseburia intestinalis, Anaerostipes caccae, Anaerostipes rhamnosivorans, Eubacterium limosum, Subdoligranulum variabile, Akkermansia muciniphila, and Bacteroides thetaiotaomicron.

More preferably, the microbiota recovery composition comprises one or more strains (at any suitable amount) of the following species: Faecalibacterium prausnitzii, Roseburia faecis, Roseburia hominis, Roseburia intestinalis, Anaerostipes caccae, Anaerostipes rhamnosivorans, Eubacterium limosum, Eubacterium sp. ARC.2, Subdoligranulum variabile, Akkermansia muciniphila, Bifidobacterium adolescentis, Bifidobacterium animalis, Bifidobacterium breve, Bifidobacterium catenulatum, Bifidobacterium crudilactis, Bifidobacterium dentium, Bifidobacterium pseudocatenulatum, Bifidobacterium stercoris, Bifidobacterium thermacidophilum, Methanobrevibacter smithii, Roseburia sp. 499, Bacteroides dorei, Bacteroides massiliensis, Bacteroides plebeius, Bacteroides sp. 35AE37, Bacteroides thetaiotaomicron, Bacteroides xylanisolvens, Lactobacillus rhamnosus, Lactococcus lactis (table 4).

The combination of all of them, or a subset of them, can be used for treatment, diagnostics, and/or any suitable purpose. One or more of the described can include and/or be associated with all, or some of the following properties: pathogen inhibition, degradation of polysaccharides, degradation of mucin, short-chain fatty acids production, production of conjugated linoleic acid, production of enterolactone, production of GABA, production of indole, and/or suitable microorganism-related functionality. In a specific example, the regression coefficient for each bacterial taxa, and some of their functions are described in the following Table 4.

In further embodiment, the microbiota recovery composition (LBP formulation as an antibiotics recovery treatment) of the present invention can further comprises at least a bacterium selected from the group consisting of Enterococcus faecium, Lactobacillus rhamnosus, Lactobacillus salivarius, Bifidobacterium adolescentis, Bifidobacterium animalis, Lactobacillus gasseri, Bifidobacterium breve, Bifidobacterium pseudocatenulatum, Lactobacillus reuteri, Lactobacillus fermentum, Pediococcus pentosaceus, Lactobacillus helveticus, Lactobacillus brevis, and Lactococcus lactis, which have been used in a probiotic.

The population of microorganisms living in the human gastrointestinal tract is commonly referred to as “microbial flora”, “gut flora”, and/or “gut microbiota”. The microbial flora of the human gut encompasses a wide variety of microorganisms that aid in digestion, the synthesis of vitamins, and creating enzymes not produced by the human body.

As used herein, the phrase “bacterial taxa of the Invention” refers to age-discriminatory bacterial taxa associated with repair of the gut microbiota. In some embodiments, one or more bacterial taxa of the invention are selected from the group listed in Table 1. Preferred combinations of bacterial taxa of the invention include, but are not limited to the combinations listed in Table 2. Combinations may also be selected by identifying bacterial taxa associated with repair of the gut microbiota that are under-represented in a subject's gut microbiota as compared to a healthy subject not consuming antibiotics.

Antibiotic consumption has strong effects on the gut microbiota through direct or indirect mechanisms. Antibiotics can disrupt the normal intestinal flora, leading to an overgrowth of harmful bacteria, such as pathogen or gastrointestinal pathogen. A healthy microbiota provides a host with multiple benefits, including colonization resistance to a broad spectrum of pathogens, essential nutrient biosynthesis and absorption, and immune stimulation that maintains a healthy gut epithelium and an appropriately controlled systemic immunity. The intestinal microbiota plays a significant role in the pathogenesis of many diseases and disorders, including a variety of pathogenic infections of the gut. For instance, subjects become more susceptible to pathogenic infections when the normal intestinal microbiota has been disturbed due to use of antibiotics. Many of these diseases and disorders are chronic conditions that significantly decrease a subject's quality of life and can be ultimately fatal. In states of dysbiosis, disrupted eubiosis, or gut imbalance which is induced or caused by antibiotic treatment (e.g., antibiotic-inducing imbalance or antibiotic-causing imbalance), microbiota functions and balance can be lost or deranged, resulting in gastrointestinal disorder such as upset stomach, constipation, diarrhea, bloating, leaky gut syndrome, hemorrhoids, inflammatory bowel disease (IBD), irritable bowel syndrome (IBS), dyspepsia, belching, rumination, abdominal pain, difficulty urinating, nausea, difficulty urinating, chest pain, skin rash, fatigue, anxiety or depression, or preferably upset stomach, constipation, diarrhea, bloating, leaky gut syndrome, hemorrhoids, IBD, IBS, dyspepsia, belching, rumination, abdominal pain or difficulty urinating.

As used herein the term “probiotics” refers to bacteria which, when consumed in sufficient amounts confer a benefit to health. As used herein the term “prebiotics” refers to substances that are non-digestible food ingredients that stimulate the growth and/or activity of bacteria in the digestive system in ways claimed to be beneficial to health. As used herein the term “synbiotics” refers to nutritional supplements or medicament for combining probiotics and prebiotics in a form of synergism. A synbiotic composition will stimulate the growth of probiotics strains present in the composition and in the indigenous microflora and to exhibit synergistic effect in vivo.

The term “subject,” as used herein, refers to a mammal, including, but not limited to, a dog, a cat, a rat, a mouse, a hamster, a mouse, a cow, a horse, a goat, a sheep, a pig, a camel, a non-human primate, and a human. In a preferred embodiment, a subject is a human.

An embodiment of the present invention is to provide a method of selecting a microorganism useful for recovering an antibiotic-inducing imbalance of gut microbiota in a subject.

Specifically, the embodiment relates to a method of selecting a microorganism useful for recovering an antibiotic-inducing imbalance of gut microbiota in a subject, comprising:

(a) receiving an aggregate set of samples from a population of subjects,

(b) obtaining a relative abundance for each bacterial taxon in the population,

(c) selecting candidate bacteria by applying the relative abundances of the bacterial taxa from step (b) to a regression model and determining the correlation between the relative abundances of a first subset of the population of subjects consuming an antibiotic, and a second subset of the population of subjects not consuming the antibiotic, and

(d) selecting bacteria useful for a microbiota recovery composition for an antibiotic-inducing imbalance of gut microbiota, by applying functional features of bacteria to the selected candidate bacteria, to exclude a bacterium having a harmful functional feature and to extract a bacterium having a beneficial functional feature from the candidate bacteria in step (c).

Fecal samples are commonly used in the art to sample gut microbiota. Methods for obtaining a fecal sample from a subject are known in the art and include, but are not limited to, rectal swab and stool collection.

Methods for extracting nucleic acids from a fecal sample are also well known in the art. The extracted nucleic acids may or may not be amplified prior to being used as an input for profiling the relative abundances of bacterial taxa, depending upon the type and sensitivity of the downstream method. When amplification is desired, nucleic acids may be amplified via polymerase chain reaction (PCR). Methods for performing PCR are well known in the art. Selection of nucleic acids or regions of nucleic acids to amplify are discussed above.

While any suitable nucleic acid known in the art may be used, one skilled in the art will appreciate that selection of a nucleic acid or region of a nucleic acid to amplify may differ by environment. In some embodiments, a nucleic acid queried is a small subunit ribosomal RNA gene. For bacterial and archaeal populations, at least one region selected from the group consisting of V1, V2, V3, V4, V5, V6, V7, V8 and V9 regions of 16S rRNA gene or 18S rRNA gene are suitable, though other suitable regions are known in the art.

The selecting candidate bacteria in the step (c), further comprises analysis of the co-occurrence probability for the first subset of the population of subjects consuming an antibiotic, or the second subset of the population of subjects not consuming the antibiotic.

Any suitable machine learning algorithm may be used to regress relative abundances of bacterial taxa against the amount of time the control subgroup of subjects not consuming antibiotics has lived at the time the gut microbiota sample was collected. In an exemplary embodiment, when antibiotics are prescribed to inhibit or eliminate bacteria that cause a specific disease, pathogen and/or opportunistic pathogens inhibited by the antibiotic are excluded from the selection of LBP candidates.

As used herein the term “pathogenic” refers to a substance or condition that has the capability to cause a disease.

As used herein the terms “gastrointestinal pathogen” or “enteropathogen” include microbes with pathogenicity for the gastrointestinal tract (from oesophagus down to rectum). It includes enterobacteria, enterococci, corynebacteria, Mycobacterium avium subspecies paratuberculosis, Brachyspira hyodysenteriae, Lawsonia intracellularis, Campylobacter, Clostridia, and others. Gastrointestinal pathogenic bacteria may include bacteria of the genus Salmonella, Shigella, Staphylococcus, Campylobacter Jejuni, Clostridium, Escherichia coli, Yersinia, Vbrio cholerae, and others.

The microbiota recovery composition can be included in a LBP formulation, for example in a probiotic formulation (and/or suitable consumable and/or therapeutic composition; etc.).

Bacterial taxa of the invention are preferably administered orally or rectally. One or more bacterial taxa of the invention may be formulated for oral or rectal administration, and may be administered alone or with an additional therapeutic agent. Non-limiting examples of additional therapeutic agents include antibiotics, antimotility agents (e.g. loperamide), antisecretory agents (e.g. racecadotril and other agents that reduce the amount of water that is released into the gut during an episode of diarrhea), bulk-forming agents (e.g. isphaghula husk, methylcellulose, sterculia, etc.) prebiotics, probiotics, synbiotics, supplemental zinc therapy, nonsteroidal anti-inflammatory drugs, mucosal protectants and adsorbents (e.g. kaolin-pectin, activated charcoal, bismuth subsalicylate, etc.).

A bacterial taxon of the invention, or a combination of bacterial taxa of the invention, is formulated to maintain a suitable level of viable cells during the formulation's shelf life and upon administration to a subject. Each bacterial taxon may be present in a wide range of amounts provided that the composition or combination delivers the effect described. The total amount of bacteria per unit dose is dependent, in part, upon the dosage form and excipients. Non-limiting examples of suitable amounts include from about 10² to about 10¹² colony forming units (cfu) of each bacterium per unit dose.

A bacterial taxon of the invention, or a combination of bacterial taxa of the invention, may be formulated into a formulation for oral or rectal administration comprising one or more bacterial taxa of the invention and one more excipients. Bacterial taxa of the invention, or a combination of bacterial taxa of the invention, may be formulated in unit dosage form as a solid, semi-solid, liquid, capsule, or powder. Usually the amount of a bacterial taxon of the invention, or a combination of bacterial taxa of the invention, is between 0.1-95% by weight of the formulation, or between 1 and 50% by weight of the formulation.

Embodiments of the method can, however, include any other suitable blocks or steps configured to facilitate reception of biological samples from subjects, processing of biological samples from subjects, analyzing data derived from biological samples, and generating models that can be used to provide customized diagnostics and/or probiotic-based therapeutics according to specific microbiome compositions and/or functional features of subjects.

Embodiments of the method and/or system can include every combination and permutation of the various system components and the various method processes, including any variants (e.g., embodiments, variations, examples, specific examples, FIGURES, etc.), where portions of embodiments of the method and/or processes described herein can be performed asynchronously (e.g., sequentially), concurrently (e.g., in parallel), or in any other suitable order by and/or using one or more instances, elements, components of, and/or other aspects of the system and/or other entities described herein.

Any of the variants described herein (e.g., embodiments, variations, examples, specific examples, FIGURES, etc.) and/or any portion of the variants described herein can be additionally or alternatively combined, aggregated, excluded, used, performed serially, performed in parallel, and/or otherwise applied.

Portions of embodiments of the method and/or system can be embodied and/or implemented at least in part as a machine configured to receive a computer-readable medium storing computer-readable instructions. The instructions can be executed by computer-executable components that can be integrated with the system.

The computer-readable medium can be stored on any suitable computer-readable media such as RAMs, ROMs, flash memory, EEPROMs, optical devices (CD or DVD), hard drives, floppy drives, or any suitable device. The computer-executable component can be a general or application specific processor, but any suitable dedicated hardware or hardware/firmware combination device can alternatively or additionally execute the instructions.

As a person skilled in the art will recognize from the previous detailed description and from the figures and claims, modifications and changes can be made to embodiments of the method, system, and/or variants without departing from the scope defined in the claims.

### MODE FOR INVENTION

In specific examples, Section 1 (below) describes specific examples of method to identify bacterial taxa as described herein, such as to be included in a LBP formulation and/or suitable therapeutic compositions. Section 2 provides specific examples of the identified species.

### Example 1: Specific Examples of Method for Identifying Taxa Affected by Antibiotics

1.1 Specific Examples of Method to Identify Bacteria that Depleted or Decreased after Antibiotic Consumption

From a list of over 64,000 Operational Taxonomic Units (OTUs), a subset was to be selected as potential candidates for inclusion in a probiotic for recover gut microbiota after a course of antibiotics. Objective criteria had to be defined for this selection. The inventors opted for selecting a subset of samples who answered a comprehensive survey, specifically claiming have either consumed or not oral antibiotics up to 3 months (and/or can be any suitable time period) before taking their gut sample (and/or can be any suitable body site sample).

The relative abundance of OTUs of these two cohorts was gathered, and statistically analyzed for detecting which microbial taxa are associated (e.g., reduced or increased) with antibiotic consumption. Two statistical approaches were used. However, any suitable number and/or type of statistical approaches can be used for statistically analyzed for detecting which microbial taxa are associated (e.g., reduced or increased) with antibiotic consumption.

As a first statistical approach, a logistic regression was conducted on CLR (centered log-ratio)-transformed relative data, using antibiotic consumption (i.e. consumer and non-consumer) as response variable, and the relative abundances of OTUs as independent variable, to obtain coefficiencies of logistic regression. CLR transformation was used to remove bias introduced in the data because of its relative nature (i.e. compositional data). The meaning of the coefficient value of logistic regression means that there is a positive relationship between the two hypotheses in case of the positive coefficient value. When the closer the coefficient value is to zero (0), the more there is no random correlation. The meaning of the coeff_log_reg value in Table 1 shows the correlation between the two groups of the antibiotic-consuming group and the antibiotic non-consuming group.

As a second statistical approach, zero-inflated negative binomial regression was conducted for each OTU's relative abundance, with antibiotic consumption behavior as factor. This analysis has the advantage that models separately zero and greater than zero abundances, and performs better than Poisson regression, because it better controls for over dispersion in the data. Additionally, it works well on count data.

Only OTUs that showed statistical difference in relative abundance (i.e. P-value equal or less than 0.05; but any suitable criteria conditions can be used) for both analyses were considered as candidates for inclusion in the probiotic (Table 1).

Selected OTUs were then annotated to its corresponding taxonomic level using SILVA taxonomy. Data can include information such as “coeff_log_reg” and “coeff_neg_bin”, which can represent the amount of change in relative abundance for each OTU estimated by the regression models under antibiotic treatment. A negative coefficient represents a decrease in abundance, whereas a positive number represents an increase in relative abundance. However, any suitable metrics can be determined for indicating effect of antibiotics on microbiome composition and/or microbiome function.

All analyses were conducted in R statistical software. Pscl and MASS packages were used for the regression analyses. Compositions package was used for performing centered log-ratio (CLR) transformation on data when necessary. However, any suitable statistical software and/or approaches and/or transformation software and/or approaches can be used.

TABLE 1
Taxa are significantly affected by antibiotic treatment

| OTU ID | Taxon | p_values_log_reg | coeff_log_reg | p_values_neg_bin | coeff_neg_bin |
|---|---|---|---|---|---|
| no_r9101581 | * * * | 0.044577261 | 1.43585382 | 0.000175934 | 1.172208664 |
| no_r91034 | * * * | 0.017008508 | 0.342716452 | 0.001509351 | 0.292699652 |
| no_r910397 | * * * | 0.013621135 | −1.545900334 | 0.016065742 | 2.766672079 |
| no_r91047 | Bacteria; Firmicutes; | 0.002051483 | 0.442950033 | 1.92E−07 | 0.623428612 |
| | Erysipelotrichia; | | | | |
| | Erysipelotrichales; | | | | |
| | Erysipelotrichaceae; | | | | |
| | Holdemania; | | | | |
| no_r91058 | Bacteria; Firmicutes; | 0.006267962 | 0.723207539 | 0.026753996 | 0.534231077 |
| | Clostridia; Clostridiales; | | | | |
| | Clostridiaceae; | | | | |
| | Clostridium; uncultured | | | | |
| | Clostridium sp. | | | | |
| no_r91086 | Bacteria;;;;;; | 0.011850968 | 0.30796753 | 0.000749079 | 0.323991527 |
| no_r9111148 | * * * | 0.019741129 | −0.824277489 | 0.048721014 | 0.434011876 |
| no_r9114757 | * * * | 0.028971651 | 1.007951538 | 0.005588773 | 0.577899302 |
| no_r911962 | Bacteria; Firmicutes; | 0.04167211 | 0.503674365 | 0.00378954 | 0.681515174 |
| | Clostridia; Clostridiales; | | | | |
| | Ruminococcaceae;; | | | | |
| no_r91206 | Bacteria; Firmicutes; | 0.001152159 | −0.450379215 | 0.042051479 | −0.401625696 |
| | Clostridia; Clostridiales;;; | | | | |
| no_r9124 | Bacteria; Firmicutes; | 0.002783306 | 0.344349992 | 0.013697663 | 0.849194891 |
| | Clostridia; Clostridiales;;; | | | | |
| no_r912825 | Bacteria; Firmicutes; | 0.01238896 | 0.842847358 | 0.007374987 | 0.533336492 |
| | Clostridia; Clostridiales;;; | | | | |
| no_r912844 | Bacteria; Firmicutes; | 0.047484509 | 0.483059227 | 0.016792553 | 1.899890751 |
| | Clostridia; Clostridiales;;; | | | | |
| no_r91325 | Bacteria; Actinobacteria; | 0.045974736 | −0.446623965 | 0.004041849 | −0.666388248 |
| | Coriobacteriia; | | | | |
| | Eggerthellales; | | | | |
| | Eggerthellaceae; | | | | |
| | Enterorhabdus; | | | | |
| | Enterorhabdus muris | | | | |
| no_r913306 | Bacteria; Firmicutes; | 0.03014051 | 1.471502357 | 0.007152161 | −2.928117627 |
| | Clostridia; Clostnidiales; | | | | |
| | Ruminococcaceae;; | | | | |
| no_r91484 | Bacteria; Firmicutes; | 0.040562142 | 0.906284619 | 6.26E−05 | −4.692765266 |
| | Clostridia; Clostridiales; | | | | |
| | Clostridiaceae;; | | | | |
| no_r91524 | Bacteria; Firmicutes; | 0.017828391 | −0.406466608 | 0.013552623 | −0.425312327 |
| | Clostridia; Clostridiales; | | | | |
| | Ruminococcaceae; | | | | |
| | Faecalibacterium; | | | | |
| no_r916098 | Bacteria | 0.00333013 | −1.461651399 | 0.044710048 | −0.353752131 |
| no_r916571 | Bacteria;;;;;; | 0.010129553 | −0.520628025 | 0.002655988 | −1.68446872 |
| no_r916590 | Bacteria; Firmicutes; | 0.030906967 | −0.555168858 | 0.015226756 | 0.491171545 |
| | Clostridia; Clostridiales; | | | | |
| | Lachnospiraceae; | | | | |
| | Anaerotignum; | | | | |
| | Anaerotignum | | | | |
| | lactatifermentans | | | | |
| no_r9199 | Bacteria; Firmicutes; | | | | |
| | Clostridla; Clostridiales;;; | | | | |
| no_r92059 | Bacteria; Firmicutes; | 0.040514505 | 0.32142331 | 0.0412519 | 0.217080538 |
| | Clostridia; Clostridiales;;; | | | | |
| no_r92072 | Bacteria; Firmicutes; | 0.007589279 | 0.872890493 | 0.01425276 | 0.654177631 |
| | Clostridia; Clostridiales; | | | | |
| | Ruminococcaceae;; | | | | |
| no_r92121 | Bacteria; Bacteroldetes; | 0.033691199 | 0.217329035 | 4.35E−06 | 0.564629946 |
| | Bacteroidia; Bacteroidales; | | | | |
| | Bacteroidaceae; | | | | |
| | Bacteroides; | | | | |
| no_r9213 | Bacteria; Firmicutes; | 0.035977712 | 0.484385219 | 0.039078774 | 0.34052352 |
| | Clostridia; Clostridiales;;; | | | | |
| no_r92152 | Bacteria; Firmicutes; | 0.030106939 | 0.600895708 | 0.019917066 | 0.53513372 |
| no_r82160 | Bacteria; Firmicutes; | | | | |
| | Erysipelotrichia; | | | | |
| | Erysipelotrichales; | | | | |
| | Erysipelotrichaceae; | | | | |
| | Holdemania; Holdemania | | | | |
| | massiliensis | | | | |
| no_r92192 | Bacteria; Firmicutes; | 0.008059445 | 0.555864903 | 5.13E−05 | 0.541331126 |
| | Clostridia; Clostridiales;;; | | | | |
| no_r92242 | Bacteria; Firmicutes; | | | | |
| | Clostridia; Clostridiales; | | | | |
| | Ruminococcaceae; | | | | |
| | Subdoligranulum; | | | | |
| | uncultured | | | | |
| | Subdoligranulum sp. | | | | |
| no_r92258 | Bacteria; Firmicutes; | 0.048180233 | 0.258944975 | 0.008532899 | 1.457304883 |
| | Clostridia; Clostridiales; | | | | |
| | Ruminococcaceae;; | | | | |
| no_r9228 | Bacteria; Firmicutes; | 0.02416536 | 0.258663537 | 0.03757321 | 1.33895803 |
| | Clostridia; Clostridiates; | | | | |
| | Lachnospiraceae;; | | | | |
| no_r92319 | Bacteria; Firmicutes; | 0.029232969 | 0.286522133 | 0.007126248 | 0.356400204 |
| | Clostridia; Clostridiales; | | | | |
| | Lachnospiraceae; | | | | |
| | Roseburia; | | | | |
| no_r92325 | Bacteria; Bacteroidetes; | 0.014883196 | 0.564070221 | 4.09E−05 | 0.872295531 |
| | Bacteroidia; Bacteroidales; | | | | |
| | Bacteroidaceae; | | | | |
| | Bacteroides; | | | | |
| no_r92370 | Bacteria; Firmicutes; | 2.91E−07 | 0.331157833 | 0.000121004 | 0.686683667 |
| | Clostridia; Clostridiales;;; | | | | |
| no_92420 | Bacteria; Firmicutes; | 0.003350243 | 0.524767607 | 0.046187332 | 0.237406202 |
| | Clostridia; Clostridiales; | | | | |
| | Ruminococcaceae;; | | | | |
| no_r92428 | Bacteria; Firmicutes; | 0.024006309 | 0.167989048 | 0.001122456 | 0.540483831 |
| | Clostridia; Clostridiales;;; | | | | |
| no_r9245 | Bacteria; Firmicutes;;;;; | 0.002895834 | 0.585510083 | 0.034989434 | 0.277182007 |
| no_r924939 | Bacteria; Firmicutes; | 0.026696064 | 0.724822103 | 0.010555245 | 2.738038813 |
| | Clostridia; Clostridiales; | | | | |
| | Clostridiaceae;; | | | | |
| no_r925 | ;;;;;; | 0.019201212 | 0.732980545 | 1.98E−05 | 5.15606987 |
| no_r92532 | Bacteria; Bacteroidetes; | 0.000536561 | 0.46708994 | 0.000677106 | 0.520106009 |
| | Bacteroidia; Bacteroidales; | | | | |
| | Bacteroidaceae; | | | | |
| | Bacteroides; uncultured | | | | |
| | Bacteroides sp. | | | | |
| no_r92538 | Bacteria; Bacteroidetes; | 0.047853522 | 0.1752038 | 2.19E−06 | 0.68032035 |
| | Bacteroidia; Bacteroidales; | | | | |
| | Bacteroidaceae; | | | | |
| | Bacteroides; uncultured | | | | |
| | Bacteroides sp. | | | | |
| no_r9254 | Bacteria; Firmicutes; | 3.13E−05 | −0.218042424 | 0.002656297 | −0.41787943 |
| | Clostridia; Clostridiates;;; | | | | |
| no_r92547 | Bacteria; Firmicutes; | 0.000711919 | 0.37075311 | 2.17E−16 | 1.100822506 |
| | Clostridia; Clostridiales;;; | | | | |
| no_r9256 | Bacteria; Actinobacteria; | 0.021136112 | −0.210897603 | 9.88E−06 | −1.495785215 |
| | Actinobacteria; | | | | |
| | Corynebacteriales; | | | | |
| | Corynebacteriaceae;; | | | | |
| no_r9271 | Bacteria; Firmicutes; | 0.016560473 | 0.1286829 | 6.36E−07 | 0.47957207 |
| | Clostridia; Clostridiales;;; | | | | |
| no_r92713 | * * * | 0.049555861 | 0.248054306 | 4.89E−07 | 2.114036154 |
| no_r9272 | Bacteria; Firmicutes: | 0.004296047 | 0.457970933 | 0.000860916 | 0.52105384 |
| | Clostridia; Clostridiales; | | | | |
| | Lachnospiraceae; | | | | |
| | Roseburia; Roseburia | | | | |
| | intestinalis | | | | |
| no_r9276 | Bacteria; Bacteroidetes; | 0.031083524 | 0.172606447 | 0.004730554 | 0.36337109 |
| | Bacteroidia; Bacteroidales; | | | | |
| | Bacteroidaceae;; | | | | |
| no_r92800 | Bacteria; Bacteroldetes; | 0.001091267 | 0.51931183 | 9.49E−07 | 0.938119248 |
| | Bacteroidia; Bacteroidales; | | | | |
| | Bacteroidaceae;; | | | | |
| no_r92980 | Bacteria; Firmicutes; | 0.023234007 | 0.467372706 | 0.011249106 | 0.57557013 |
| | Clostridia; Clostridiales; | | | | |
| | Ruminococcaceae;; | | | | |
| no_r92999 | Bacteria; Firmnicules; | 0.01962132 | 1.22591266 | 0.004375313 | 0.682883311 |
| | Clostridia; Clostridiales; | | | | |
| | Eubacteriaceae;; | | | | |
| no_r93050 | Bacteria; Firmicutes; | 0.025249545 | 0.364713282 | 0.000328142 | 0.404419447 |
| | Clostridia; Clostridiales;;; | | | | |
| no_r93079 | Bacteria; Firmicutes; | 5.95E−06 | 0.279727134 | 5.27E−06 | 1.039301259 |
| | Clostridia; Clostridiales; | | | | |
| | Lachnospiraceae; Blautia; | | | | |
| no_r93102 | Bacteria; Firmicutes; | 0.00249066 | 0.573206108 | 0.003879667 | 2.256557291 |
| | Clostridia; Clostridiales; | | | | |
| | Clostridiaceae;; | | | | |
| no_r93105 | Bacteria; Bacteroidetes; | 0.03010686 | 0.668077603 | 2.73E−05 | 2.077872251 |
| | Bacteroidia; Bacteroidales; | | | | |
| | Bacteroidaceae; | | | | |
| | Bacteroides; | | | | |
| no_r93139 | Bacteria; Firmicutes; | 0.000693706 | 0.355149253 | 2.06E−06 | 1.029231896 |
| | Clostridia; Clostridiales;;; | | | | |
| no_r9316 | Sacteria; Firmicutes; | 0.000174036 | 0.192149282 | 0.042657514 | 0.417845559 |
| | Clostridia; Clostridiales;; | | | | |
| no_r93331 | Bacteria; Firmicules; | 0.025258342 | 0.347726122 | 0.000138413 | 0.551532863 |
| | Clostridia; Clostridiales; | | | | |
| | Clostridiaceae;; | | | | |
| no_r9338 | Bacteria; Firmicutes; | 0.022784829 | 0.469501822 | 0.002832426 | 0.773713889 |
| | Clostridia; Clostridiales;;; | | | | |
| no_r9341 | Bacteria; Firmicutes; | 1.86E−05 | −0.246206874 | 0.032325627 | −0.490513798 |
| | Clostridia; Clostridiales; | | | | |
| | Christensenellaceae;; | | | | |
| no_r93430 | Bacteria; Firmicutes; | 0.014662248 | 0.81519419 | 0.000394265 | 1.12781641 |
| | Clostridia; Clostridiales; | | | | |
| | Ruminococcaceae; | | | | |
| | Faecalibacterium; | | | | |
| no_r9344 | Bacteria; Firmicules; | 0.014948769 | 0.339015284 | 0.015041804 | 0.757790768 |
| | Clostridia; Clostridiales; | | | | |
| | Clostridiales Family XIII. | | | | |
| | Incertae Sedis; | | | | |
| | Mobilibacterium; | | | | |
| | Mobilibacterium | | | | |
| | timonense | | | | |
| no_r93488 | Bacteria; Firmicutes; | 0.00286211 | 1.44343568 | 0.001549438 | 0.751233483 |
| | Clostridia; Clostridiales; | | | | |
| | Lachnospiraceae;; | | | | |
| no_r9357 | Bacteria; Firmicutes; | 0.001100407 | 0.259939298 | 0.000676912 | 0.491664759 |
| | Clostridia; Clostridiates;;; | | | | |
| no_r936184 | Bacteria; Planctomycetes; | 0.016904715 | 0.86928943 | 0.008732235 | 2.672001068 |
| | Planctomycetia; | | | | |
| | Planctomycetales; | | | | |
| | Planctomycetaceae:; | | | | |
| no_r9364 | Bacteria; Firmicutes; | 0.021368536 | −0.211030958 | 0.032053397 | −0.791787715 |
| | Clostridia; Clostridiales; | | | | |
| | Ruminococcaceae; | | | | |
| | Ruminococcus; uncultured | | | | |
| | Ruminococcus sp. | | | | |
| no_93682 | Bacteria;;;;;; | 0.008268858 | 0.637790729 | 0.010328431 | 0.55677456 |
| no_9372 | Bacteria; Firmicutes; | 0.036713998 | 0.089045093 | 0.024654845 | 0.331830525 |
| | Clostridia; Clostridiales;; | | | | |
| no_r93763 | Bacteria; Firmicutes; | 0.002032043 | 0.668889323 | 0.018709634 | 0.430255479 |
| | Clostridia; Clostridiales; | | | | |
| | Lachnospiraceae;: | | | | |
| no_r9377 | Bacteria; Firmicutes; | 0.003671835 | -0.076803425 | 0.021453964 | −0.291977865 |
| | Clostridia; Clostridiales; | | | | |
| | Ruminococcaceae; | | | | |
| | Faecalibacterium; | | | | |
| no_r9378 | Bacteria;;;;;; | 0.0117519 | 0.339463417 | 5.00E−05 | 0.590382677 |
| no_r9379 | Bacteria; Firmicutes; | 0.003018787 | 0.557525504 | 0.028140986 | 0.320255057 |
| | Clostridia; Clostridiales;; | | | | |
| no_r93797 | Bacteria; Firmicutes; | 0.002732294 | −0.85400268 | 0.016051188 | −0.53887261 |
| | Bacilli; Lactobacillales; | | | | |
| | Streptococcaceae; | | | | |
| | Streptococcus; uncultured | | | | |
| | Streptococcus sp. | | | | |
| no_r93815 | * * * | 0.01214224 | 0.397436632 | 0.001459682 | 1.812044006 |
| no_r93874 | Bacteria; Firmicutes; | 0.009478532 | 0.669100379 | 0.008207885 | 1.00868788 |
| | Clostridia; Clostridiales; | | | | |
| | Lachnospiraceae; | | | | |
| | Roseburia; | | | | |
| no_r94049 | Bacteria; Firmicutes; | 0.043604144 | 0.266755412 | 0.004993952 | 0.339455627 |
| | Clostridia; Clostridiales;;; | | | | |
| no_r9410 | Bacteria; Firmicutes; | 0.002670275 | −0.1850396 | 0.001496305 | −0.670505858 |
| | Clostridia; Clostridiales; | | | | |
| | Clostridlaceae; | | | | |
| | Clostridium; uncultured | | | | |
| | Clostridium sp. | | | | |
| no_r94249 | Bacteria; Firmicutes; | 0.022423709 | 0.298912978 | 0.019457952 | 0.410988455 |
| | Clostridia; Clostridiales; | | | | |
| | Lachnospiraceae; | | | | |
| | Lachnoclostridium; | | | | |
| | [Clostridium] aldenense | | | | |
| no_r94337 | Bacteria; Firmicutes; | 0.005011024 | 0.727593553 | 0.004102351 | 1.274662384 |
| | Clostridia; Clostridiales;;; | | | | |
| no_r94351 | Bacieria; Firmicutes; | 0.010255384 | 0.252557925 | 3.24E−06 | 0.996067593 |
| | Erysipelotrichia; | | | | |
| | Erysipelotrichales; | | | | |
| | Erysipelotrichaceae;; | | | | |
| no_r9445 | Bacteria; Firmicutes; | 1.00E−05 | 0.252846804 | 5.88E−09 | 0.780594771 |
| | Clostridia; Clostridiales;;; | | | | |
| no_r9459 | Bacteria; Firmicutes; | 0.044517619 | 0.443988646 | 0.00083191 | 2.331385557 |
| | Clostridia; Clostridiales;;; | | | | |
| no_r9494 | Bacteria; Firmicutes; | 0.003936102 | 0.30378764 | 0.002561373 | 0.461369423 |
| | Clostridia; Clostridiales; | | | | |
| | Lachnospiraceae;; | | | | |
| no_r95029 | Bacteria; Firmicutes; | 0.004094096 | 0.61244572 | 0.000150246 | 0.566055695 |
| | Clostridia; Clostridiales; | | | | |
| | Lachnospiraceae; | | | | |
| | Roseburia; | | | | |
| no_r95455 | Bacteria; Firmicutes; | 0.004815276 | 0.468733531 | 0.000272894 | 0.502652024 |
| | Clostridia; Clostridiales; | | | | |
| | Clostridiaceae; | | | | |
| | Massilloclostridium; | | | | |
| | Massilloclostridium coli | | | | |
| no_r95657 | Bacteria; Firmicutes; | 0.003110317 | −0.370472121 | 0.009196922 | −0.857833393 |
| | Clostridia; Clostridiales; | | | | |
| | Ruminococcaceae;; | | | | |
| no_r9576 | * * * | 0.022979252 | 0.18812815 | 3.00E−05 | 1.196445625 |
| no_r958 | Bacteria; Firmicutes; | 0.000121669 | −0.156585613 | 0.000192901 | −0.369495634 |
| | Clostridia; Clostridiales; | | | | |
| | Lachnospiraceae;; | | | | |
| no_r96210 | Bacteria; Proteobacteria; | 0.032526242 | −0.696100896 | 0.022850368 | −0.427038087 |
| | Deltaproteobacteria; | | | | |
| | Desulfovibrionales; | | | | |
| | Desulfovibrionaceae; | | | | |
| | Bilophila; | | | | |
| no_r96262 | Bacteria; Firmicutes; | 0.017580506 | −0.516204703 | 0.003889989 | −1.435427295 |
| | Clostridia; Clostridiales; | | | | |
| | Ruminococcaceae;; | | | | |
| no_r964 | Bacteria; Firmicutes; | 0.00626906 | −0.160300318 | 0.038279564 | 0.196902842 |
| | Clostridia; Clostridiates;;; | | | | |
| no_r970 | Bacteria; Firmicutes; | 0.014418126 | −0.10001374 | 0.035666691 | 0.248717961 |
| | Clostridia; Clostridiales;;; | | | | |
| no_97396 | Bacteria; Bacteroidetes; | 0.01195232 | 0.852373508 | 2.08E-06 | 1.050073169 |
| | Bacteroidia; Bacteroidales; | | | | |
| | Bacteroidaceae;; | | | | |
| no_97450 | Bacteria; Bacteroidetes; | 0.021092692 | 0.876795504 | 0.016751134 | 0.742192367 |
| | Bacteroidia; Bacteroidales; | | | | |
| | Bacteroidaceae; | | | | |
| | Bacteroides; | | | | |
| no_r97783 | Bacteria;;;;;; | 0.021702401 | −0.57953486 | 1.05E-05 | −1.889148258 |
| no_r98366 | Bacteria; Firmicutes; | 0.027513406 | 0.699946429 | 0.012945001 | 0.566335113 |
| | Clostridia; Clostridiales; | | | | |
| | Eubacteriaceae; | | | | |
| | Eubacterium; | | | | |
| | [Eubacterium] eligens | | | | |
| no_r9935 | Bacteria; Fusobacteria; | 0.009170889 | 0.707226856 | 0.00369705 | 0.741244151 |
| | Fusobacteriia; | | | | |
| | Fusobacteriales; | | | | |
| | Leptotrichiaceae; | | | | |
| | Leptotrichia; | | | | |
| no_r997 | Bacteria; Firmicutes; | 0.020310778 | 0.239480975 | 1.25E−06 | 1.004276196 |
| | Clostridia; Clostridiales;;; | | | | |
| r9_10179 | Bacteria; Firmicutes; | 0.030081718 | 0.144317086 | 0.012667455 | 0.545577916 |
| | Clostridia; Clostridiales; | | | | |
| | Lachnospiraceae; Blautia; | | | | |
| | Blautia sp. N6H1-15 | | | | |
| r9_1091 | Bacteria; Firmicutes; | 2.99E-06 | −0.129714684 | 0.002531437 | −0.214039584 |
| | Clostridia; Clostridiales;;; | | | | |
| r9_109177 | * * * | 0.046879516 | 0.516131902 | 7.09E−05 | 0.762189348 |
| r9_109518 | * * * | 0.043867549 | −0.386385598 | 0.011442487 | 0.208594608 |
| r9_1103 | Bacteria; Firmicutes; | 8.15E−11 | 0.283173158 | 3.66E−11 | 0.582116894 |
| | Clostridia; Clostridiales;;; | | | | |
| r9_1106 | Bacteria; Firmicutes; | 7.33E-05 | −0.167779414 | 0.042549565 | 0.101841927 |
| | Clostridia; Clostridiales;;; | | | | |
| r9_1114 | Bacteria; Bacteroidetes; | 0.017715438 | 0.284769839 | 0.002195923 | 0.448611153 |
| | Bacteroidia; Bacteroidales; | | | | |
| | Bacteroidaceae; | | | | |
| | Bacteroides; | | | | |
| r9_11344 | Bacteria; Firmicutes; | 0.000879253 | −0.394342933 | 0.002277071 | −0.582892936 |
| | Clostridia; Clostridiales; | | | | |
| | Lachnospiraceae;; | | | | |
| r9_1138 | Bacteria; Firmicutes; | 2.66E−11 | −0.143367725 | 0.004393831 | −0.272307396 |
| | Clostridia; Clostridiales; | | | | |
| | Ruminococcaceae; | | | | |
| | Faecalibacterium; | | | | |
| r9_11433 | Bacteria; Firmicutes; | 0.000757944 | −0.101572029 | 0.036831015 | 0.213440532 |
| | Clostridia; Clostridiales; | | | | |
| | Lachnospiraceae; | | | | |
| | Roseburia; Roseburia | | | | |
| | intestinalis | | | | |
| r9_1147 | *** | 0.040923473 | 0.651340214 | 0.041266881 | −1.812780871 |
| r9_1188 | Bacteria; Firmicutes; | 0.009720546 | −0.257626809 | 3.42E−08 | −1.157413097 |
| | Clostridia; Clostridiales; | | | | |
| | Clostridiaceae; | | | | |
| | Clostridium; uncultured | | | | |
| | Clostridium sp. | | | | |
| r9_119647 | Bacteria; Firmicutes; | 0.018016237 | 0.474602173 | 0.000195223 | 1.738067098 |
| | Clostridia; Clostridiales; | | | | |
| | Lachnospiraceae;; | | | | |
| r9_123671 | Bacteria; Bacteroidetes; | 0.018584608 | 0.872819908 | 0.001381238 | 0.526065043 |
| | Bacteroidia; Bacteroidales; | | | | |
| | Bacteroidaceae; | | | | |
| | Bacteroides; | | | | |
| r9_1237 | Bacteria; Firmicutes; | 0.028298112 | 0.517328068 | 0.021083117 | -0.313060521 |
| | Clostridia; Clostridiales; | | | | |
| | Ruminococcaceae; | | | | |
| | Faecalibacterium; | | | | |
| r9_124113 | Bacteria; | 0.001584855 | 0.651097649 | 0.000242891 | 0.570812775 |
| | Proteobacteria;;;;;; | | | | |
| r9_12514 | Bacteria; Firmicutes; | 0.017026524 | 0.592065549 | 0.013703419 | −0.419542734 |
| | Clostridia; Clostridiales; | | | | |
| | Eubacteriaceae;; | | | | |
| r9_128592 | * * * | 0.019634677 | 2.29256585 | 0.044426347 | −2.425463369 |
| r9_133368 | Bacteria; Firmicutes; | 0.022166456 | 1.063065243 | 0.034092203 | −2.083831545 |
| | Clostridia; Clostridiales; | | | | |
| | Lachnospiraceae;; | | | | |
| 19_134201 | Bacteria;;;;;; | 0.017073462 | 0.388509209 | 6.37E−05 | 0.50530435 |
| r9_138972 | Bacteria; Firmicutes; | 0.045690016 | 0.256195991 | 0.008294802 | 0.685961485 |
| | Clostridia; Clostridiales;;; | | | | |
| r9_1409 | * * * | 0.006660074 | 0.265674196 | 1.78E−05 | 0.610107525 |
| r9_141021 | Bacteria; Firmicutes; | 0.020840906 | −0.970628847 | 2.84E−05 | −4.885808312 |
| | Clostridia; Clostridiales; | | | | |
| | Ruminococcaceae;; | | | | |
| r9_141084 | | 0.043007229 | 0.492712225 | 0.019309559 | 0.389881977 |
| r9_144042 | Bacteria; Firmicutes; | 0.017566847 | 0.390629542 | 0.001310433 | 0.355784807 |
| | Clostridia; Clostridiales; | | | | |
| | Lachnospiraceae;; | | | | |
| r9_14678 | Bacteria; Firmicutes; | 4.95E−05 | 0.191048676 | 0.034300431 | 0.305898039 |
| | Clostridia; Clostridiales; | | | | |
| | Clostridiaceae; | | | | |
| | Clostridium; Clostridium | | | | |
| | sp. AT4 | | | | |
| r9_151195 | * * * | 0.017182053 | 0.751457375 | 0.023378965 | 0.392196638 |
| r9_1529 | Bacteria; Firmicutes; | 0.019954396 | 0.106799548 | 0.009141313 | 0.538249863 |
| | Negativicutes; | | | | |
| | Acidaminococcales; | | | | |
| | Acidaminococcaceae; | | | | |
| | Acidaminococous; | | | | |
| r9_153019 | * * * | 0.016691978 | 1.868917259 | 0.004258858 | −3.199799458 |
| r9_154708 | Bacteria; Actinobacteria; | 0.033835339 | −2.506671128 | 0.000186101 | −4.681946689 |
| | Coriobacteriia; | | | | |
| | Corobacteriales; | | | | |
| | Atopoblaceae; | | | | |
| | Libanicoccus; | | | | |
| | Libanicoccus massiliensis | | | | |
| r9_160359 | Bacteria; Firmicutes; | 0.026699336 | 0.458107457 | 0.016737962 | −0.351139442 |
| | Clostridia; Clostridiales;;; | | | | |
| r9_161832 | Bacteria; Firmicutes; | 0.00041345 | 0.668173997 | 0.000601454 | 0.480840514 |
| | Clostridia; Clostridiales;;; | | | | |
| r9_16245 | Bacteria; Firmicutes; | 0.014622367 | −0.388063288 | 0.008054207 | −0.708567691 |
| | Clostridia; Clostridiales; | | | | |
| | Lachnospiraceae;; | | | | |
| r9_1625 | Bacteria; Firmicutes; | 7.60E−07 | 0.174628523 | 0.006480365 | 0.376605665 |
| | Clostridia; Clostridiales;;; | | | | |
| r9_162510 | Bacteria; Firmicutes; | 0.039969179 | −0.615439749 | 0.001301095 | −1.287660853 |
| | Clostridia; Clostridiales;;; | | | | |
| r9_1681 | Bacteria; Firmicutes; | 0.000826697 | −0.086283372 | 0.000595097 | 0.223463366 |
| | Clostridia; Clostridiales; | | | | |
| | Lachnospiraceae; Blautia; | | | | |
| r9_16875 | Bacteria; Firmicutes; | 0.000523784 | 0.198388158 | 1.24E−05 | 0.399283702 |
| | Clostridia; Clostridiales;;; | | | | |
| r9_17260 | Bacteria; Firmicutes; | 0.000832855 | −0.231546281 | 0.031995141 | −0.386467277 |
| | Clostridia; Clostridiales; | | | | |
| | Ruminococcaceae; | | | | |
| | Ruminococcus; uncultured | | | | |
| | Ruminococcus sp. | | | | |
| r9_17334 | Bacteria; Firmicutes; | 0.029747916 | 0.503416092 | 2.69E−07 | 3.981950684 |
| | Bacilli;;; | | | | |
| r9_17356 | Bacteria; Firmicutes; | 0.014390739 | 0.275040546 | 1.63E−05 | 0.927081245 |
| | Clostridia; Clostridiales; | | | | |
| | Clostridiaceae;; | | | | |
| r9_174195 | Bacteria; Bacteroldetes; | 0.01049401 | 0.621949578 | 0.009861003 | 0.588320507 |
| | Bacteroidia; Bacteroidales; | | | | |
| | Bacteroidaceae;; | | | | |
| r9_1742 | Bacteria; Firmicutes; | 0.024792458 | 0.305069121 | 0.016567731 | 0.196042273 |
| | Clostridia; Clostridiates;;; | | | | |
| r9_17421 | Bacteria; Firmicutes; | 0.000430528 | −0.631647543 | 0.00151514 | −0.501439449 |
| | Clostridia; Closiridiales; | | | | |
| | Ruminococcaceae; | | | | |
| | Gemmiger; Gemmiger | | | | |
| | formicilis | | | | |
| r9_1756 | Bacteria; Firmicutes; | 0.014781366 | −0.856153803 | 0.000116101 | −1.162236087 |
| | Clostridia; Clostridiales; | | | | |
| | Lachnospiraceae; | | | | |
| | Coprococcus; | | | | |
| r9_1763 | * * * | 0.028021609 | −1.03035132 | 0.036958198 | −0.338486825 |
| r9_176950 | Bacteria; Firmicutes; | 0.033504252 | 0.537699823 | 0.001048979 | 0.486952006 |
| | Clostridia; Clostridiates;;; | | | | |
| r9_1773 | Bacteria; Firmicutes; | 0.005102217 | −0.088779155 | 0.000614987 | 0.305487976 |
| | Clostridia; Closirdiales;;; | | | | |
| r9_1774 | Bacteria; Firmicutes; | 0.011980387 | 0.341769267 | 0.024890692 | 0.219967068 |
| | Clostridia; Clostridiales; | | | | |
| | Ruminococcaceae;; | | | | |
| r9_1808 | Bacteria; Firmicutes; | 9.68E−05 | 0.781031678 | 0.014552048 | 0.327310415 |
| | Clostridia; Clostridiales; | | | | |
| | Ruminococcaceae; | | | | |
| | Faecalibacterium; | | | | |
| r9_1885 | Bacteria; Firmicutes; | 0.003086349 | −0.971816653 | 9.97E−05 | −0.815660078 |
| | Clostridia; Clostridiales; | | | | |
| | Lachnospiraceae;; | | | | |
| r9_1917 | Bacteria; Firmicutes;;;;; | 0.003471486 | 0.192518627 | 2.93E−13 | 0.989316466 |
| r9_19185 | Bacteria; Firmicutes; | 0.012406613 | 0.660467899 | 0.040984392 | 2.274694544 |
| | Negativicules; | | | | |
| | Veillonellales; | | | | |
| | Veillonellaceae;; | | | | |
| 191931 | Bacteria; Firmicutes; | 0.000242284 | −0.123102748 | 0.036219537 | 0.132160232 |
| | Clostridia; Clostridiates;;; | | | | |
| r9_19514 | Bacteria; Firmicutes; | 0.001231127 | 0.555387226 | 0.010425862 | −0.933636746 |
| | Clostridia; Clostridiales;;; | | | | |
| r9_197327 | Bacteria; Bacteroidetes; | 0.023722155 | 0.498225318 | 0.000676543 | 0.666009217 |
| | Bacteroidia; Bacteroidales; | | | | |
| | Bacteroidaceae; | | | | |
| | Bacteroides; | | | | |
| r9_20089 | Bacteria; Firmicutes; | 5.16E−11 | 0.282320665 | 2.95E−09 | 0.678691308 |
| | Clostridia; Clostridiales;;; | | | | |
| r9_203202 | Bacteria; Bacteroidetes; | 0.047555071 | 0.47096887 | 0.001154857 | 0.820248345 |
| | Bacteroidia; Bacteroidales; | | | | |
| | Bacteroidaceae; | | | | |
| | Bacroides; | | | | |
| r9_204046 | Bacteria; Bacteroidetes; | | | | |
| | Bacteroidia; Bacteroidales; | | | | |
| | Bacteroidaceae;; | | | | |
| r9_2063 | Bacteria; Bacteroidetes; | 0.026241242 | 1.023219044 | 0.000102553 | 1.128730807 |
| | Bacteroidia; Bacteroidales; | | | | |
| | Bacteroidaceae; | | | | |
| | Bacteroides; | | | | |
| r9_2077 | Bacteria; Firmicutes; | 8.47E−05 | 1.014917236 | 0.047263101 | 0.327203078 |
| | Clostridia; Clostridiales;;; | | | | |
| r9_207941 | Bacteria; | 0.008735883 | 0.594078424 | 0.001183789 | 0.672755538 |
| | Proteobacteria;;;;; | | | | |
| r9_2085 | Bacteria; Firmicutes; | 0.000517141 | 0.335146985 | 5.18E−06 | 0.34737461 |
| | Clostridia; Clostridiales;;; | | | | |
| r9_208677 | | 0.020921073 | 1.359571135 | 0.032182526 | −2.248499664 |
| r9_208903 | | 0.011397316 | 0.97309211 | 0.039043354 | 0.509726799 |
| r9_20950 | Bacteria; Firmicutes; | 0.012698816 | 0.206591958 | 3.46E−05 | 0.816409463 |
| | Clostridla; Clostridiales;;; | | | | |
| r9_212 | Bacteria;;;;;; | 0.011839648 | 0.133445435 | 1.23E−06 | 0.491410011 |
| r9_2155 | Bacteria; Firmicutes; | 8.72E−05 | 0.127967455 | 5.47E−08 | 0.529132939 |
| | Clostridla; Clostridiales;;; | | | | |
| r9_21657 | Bacteria; Firmicutes; | 0.011336911 | 0.362455758 | 3.15E−07 | 0.777640764 |
| | Clostridia; Clostridiales; | | | | |
| | Ruminococcaceae; | | | | |
| | Subdoligranulum; | | | | |
| | uncultured | | | | |
| | Subdoligranulum sp. | | | | |
| r9_2168 | Bacteria; Firmicutes; | 0.008366759 | 0.452806194 | 0.008864773 | 0.352329856 |
| | Clostridia; Clostridiales; | | | | |
| | Clostridiaceae;; | | | | |
| r9_2169 | Bacteria; Firmicutes; | 0.008425044 | −0.054537954 | 0.008540015 | 0.153062211 |
| | Clostridia; Clostridiales;;; | | | | |
| r9_2196 | Bacteria; Firmicutes; | 0.043005524 | −0.171426698 | 0.000479317 | −0.423507148 |
| | Clostridia; Clostridiales; | | | | |
| | Lachnospiraceae;; | | | | |
| r9_222600 | * * * | 0.032613593 | 0.665271425 | 9.21E−05 | 0.922732015 |
| r9_222624 | Bacteria; Firmicutes; | 0.009044878 | 0.598204378 | 0.00743473 | 0.670450866 |
| | Clostridia; Clostridiales; | | | | |
| | Lachnospiraceae;; | | | | |
| r9_2275 | Bacteria; Firmicutes; | 3.27E−11 | −0.227182616 | 0.031226296 | −0.156312212 |
| | Clostridia; Clostridiales; | | | | |
| | Clostridiaceae; | | | | |
| | Clostridium; uncultured | | | | |
| | Clostridium sp. | | | | |
| r9_229763 | Bacteria; Firmicutes; | 0.007048537 | 1.063526976 | 0.000691912 | 0.875283341 |
| | Clostridia; Clostridiales; | | | | |
| | Clostridiaceae; | | | | |
| | Clostridium; Clostridium | | | | |
| | sp. Marseille-P2538 | | | | |
| r9_2302 | Bacteria; Bacteroidetes; | 0.011388437 | 0.368537125 | 0.011132959 | 0.395419448 |
| | Bacteroidia; Bacteroidales; | | | | |
| | Bacteroidaceae; | | | | |
| | Bacteroides; | | | | |
| r9_231 | Bacteria; Firmicutes; | 0.041040136 | 0.283346892 | 0.024733808 | 0.214648843 |
| | Clostridia; Clostridiales;;; | | | | |
| r9_232920 | Bacteria; Bacteroidetes; | 0.013556055 | 0.80637998 | 0.005361293 | 0.552881651 |
| | Bacteroidia; Bacteroidales; | | | | |
| | Bacteroidaceae; | | | | |
| | Bacteroides; | | | | |
| r9_2334 | Bacteria; Firmicutes; | 0.008370781 | 0.32590038 | 0.008844072 | 0.331036595 |
| | Clostridia; Clostridiales; | | | | |
| | Clostridiaceae; | | | | |
| | Clostridium; uncultured | | | | |
| | Clostridium sp. | | | | |
| r9_234 | Bacteria; Firmicutes; | 0.045630121 | 0.399421885 | 0.014370237 | 0.753520404 |
| | Tissierellia; Tissierellales; | | | | |
| | Peptoniphilaceae; | | | | |
| | Anaerococcus; | | | | |
| r9_2396 | Bacteria; Bacteroidetes; | 0.048528627 | 0.124459381 | 0.000977096 | 0.432727645 |
| | Bacteroidia; Bacteroidales; | | | | |
| | Bacteroidaceae; | | | | |
| | Bacteroides; uncultured | | | | |
| | Bacteroides sp. | | | | |
| r9_242180 | Bacteria; Firmicutes; | 0.033159644 | 0.720652809 | 0.024169831 | 0.504579494 |
| | Clostridia; Clostridiales; | | | | |
| | Clostridlaceae;; | | | | |
| r9_243891 | Bacteria; Firmicutes; | 0.041778747 | −3.115982799 | 0.007241394 | −3.533425009 |
| | Erysipelotrichia; | | | | |
| | Erysipelotrichales; | | | | |
| | Erysipelotrichaceae;; | | | | |
| r9_2488 | Bacteria; Firmicutes;;;;; | 4.06E−14 | −0.235338032 | 0.025333343 | −0.189702308 |
| r9_25411 | Bacteria; Firmicutes; | 0.020261569 | −0.857625928 | 8.83E−07 | −2.209807167 |
| | Baciili; Lactobacillales; | | | | |
| | Aerococcaceae; | | | | |
| | Facidamia; | | | | |
| r9_2564 | Bacteria; Firmicutes; | 0.001444095 | 0.526292963 | 1.15E−05 | 0.656236276 |
| | Clostridia; Clostridiales; | | | | |
| | Ruminococcaceae;; | | | | |
| r9_2567 | Bacteria; Firmicutes; | 0.001386342 | 0.2818344 | 1.41E−07 | 0.754470535 |
| | Clostridia; Clostridiales;;; | | | | |
| r9_257261 | Bacteria;;;;;; | 0.047419923 | 2.062107842 | 0.006342788 | −3.450836732 |
| r9_2573 | * * * | 0.024493058 | 1.649273818 | 0.014648944 | −2.891315605 |
| r9_2609 | Bacteria; Firmicutes; | 0.000274767 | 0.207525761 | 7.35E−14 | 0.926023746 |
| | Clostridia; Clostridiates;;; | | | | |
| r9_2653 | Bacteria; Firmicutes; | 0.007941937 | 0.233209185 | 0.0206218 | 0.420313186 |
| | Clostridia; Clostridiales; | | | | |
| | Ruminococcaceae;; | | | | |
| r9_2664 | Bacteria; Firmicutes; | 0.035176178 | 0.492638239 | 0.000582118 | 0.616120569 |
| | Erysipelotrichia; | | | | |
| | Erysipelotrichales; | | | | |
| | Erysipelotrichaceae; | | | | |
| | Turicibacter; | | | | |
| r9_269765 | Bacteria;;;;;; | 0.008724759 | 0.532764932 | 2.30E−06 | 0.720371925 |
| r9_2715 | Bacteria; Firmicutes; | 0.000589665 | 0.278520853 | 6.21E−07 | 0.529701176 |
| | Clostridia; Clostridiales; | | | | |
| | Lachnospiraceae;; | | | | |
| r9_27182 | Bacteria; Firmicutes; | 0.032637265 | 0.40124931 | 0.005227779 | 2.024416274 |
| | Bacilli;;;; | | | | |
| r9_272074 | Bacteria; Bacteroidetes; | 0.040126207 | 0.33673777 | 3.54E−07 | 1.178350996 |
| | Bacteroidia; Bacteroidales; | | | | |
| | Bacteroidaceae;; | | | | |
| r9_2751 | Bacteria; | 0.039944192 | −0.05639799 | 0.004519107 | 0.320109092 |
| | Proteobacteria;;;;; | | | | |
| r9_27573 | Bacteria; Firmicutes; | 0.043451467 | 0.171495158 | 0.011803668 | 0.361341119 |
| | Clostridia; Clostridiales; | | | | |
| | Lachnospiraceae; | | | | |
| | Lachnoclostridium; | | | | |
| | [Clostridium] aldenense | | | | |
| r9_276217 | | 0.036916133 | 1.93135388 | 0.006637951 | −3.303449873 |
| r9_2779 | Bacteria;;;;;; | 0.00836614 | 0.456129743 | 4.88E−05 | 1.767909061 |
| r9_2816 | Bacteria; Firmicutes; | 0.019334772 | 5.352729246 | 0.042276189 | 0.18861352 |
| | Clostridia; Clostridiales;; | | | | |
| r9_281673 | Bacteria; Firmicutes; | 0.018376536 | -0.981642782 | 0.017749134 | −0.983607087 |
| | Clostridia; Clostridiales; | | | | |
| | Ruminococcaceae;; | | | | |
| r9_2834 | Bacteria; Firmicutes; | 3.05E−09 | -0.437464847 | 3.985−05 | −0.308487198 |
| | Clostridia; Clostridiales; | | | | |
| | Ruminococcaceae; | | | | |
| | Faecalibacterium; | | | | |
| r9_2848 | Bacteria;;;;;; | 0.027136183 | 0.47214394 | 0.00119169 | 2.446355788 |
| r9_2876 | Bacteria; Firmicutes; | 2.21E−05 | 0.169033811 | 1.04E−17 | 0.61963392 |
| | Clostridia; Clostridiales;;; | | | | |
| r9_2915 | Bacteria; Fimmicutes; | 0.00376439 | 0.416627298 | 0.010197711 | 0.27502963 |
| | Clostridia; Clostridiales; | | | | |
| | Clostridiales Family XIII. | | | | |
| | Incertae Sedis;; | | | | |
| r9_29699 | Bacteria; Firmicutes; | 0.028248943 | 0.165336923 | 2.17E−08 | 0.734591949 |
| | Clostridia; Clostridiales;;; | | | | |
| r9_3029 | Bacteria; Firmicutes; | 0.003939654 | 0.306659792 | 0.005589001 | 0.296646948 |
| | Clostridia; Clostridiales; | | | | |
| | Ruminococcaceae;; | | | | |
| r9_3063 | Bacteria; Firmicutes; | 0.030397406 | −0.216923292 | 0.000519515 | 0.774447098 |
| | Clostridia; Clostridiales; | | | | |
| | Ruminococcaceae;; | | | | |
| r9_31011 | Bacteria; Firmicutes; | 0.030875905 | −0.230784935 | 0.030302129 | 0.358435691 |
| | Clostridia; Clostridiales; | | | | |
| | Ruminococcaceae;; | | | | |
| r9_3181 | Bacteria; Firmicutes; | | | | |
| | Clostridia; Clostridiales;; | | | | |
| r9_319629 | Bacteria; Firmicutes; | 0.009194363 | −0.771064131 | 0.016118579 | −0.467537388 |
| | Clostridia; Clostridiales; | | | | |
| | Lachnospiraceae; Blautia; | | | | |
| r9_321517 | Bacteria;;;;;; | 0.013748576 | 0.860703066 | 0.004022318 | 0.666138563 |
| r9_325193 | Bacteria; Bacteroidetes; | 0.040374629 | −0.777288854 | 0.027521251 | −0.281925685 |
| | Bacteroidia; Bacteroidales; | | | | |
| | Odoribacteraceae; | | | | |
| | Odoribacter; | | | | |
| r9_3292 | Bacteria; Firmicutes; | 0.044336095 | 0.240524533 | 0.039411322 | −0.170198141 |
| | Clostridia; Clostridiales; | | | | |
| | Ruminococcaceae; | | | | |
| | Sporobacter; | | | | |
| | Sporobacter termitidis | | | | |
| r9_331702 | Bacteria; Firmicutes; | 0.011092892 | −0.961704406 | 0.007752603 | −0.667171018 |
| | Erysipelotrichia; | | | | |
| | Erysipelotrichales; | | | | |
| | Erysipelotrichaceae; | | | | |
| | Erysipelatoclostridium; | | | | |
| | Erysipelatoclostridium | | | | |
| | ramosum | | | | |
| r9_332186 | Bacteria; Firmicutes; | 0.033128527 | -0.801812979 | 0.002777726 | −0.594866111 |
| | Clostridia; Clostridiales;;; | | | | |
| r9_338230 | * * * | 0.015737966 | 1.614834671 | 0.038408956 | −2.180648661 |
| r9_345013 | Bacteria; Bacteroidetes; | 0.032055391 | 0.484427151 | 1.00E−07 | 1.42671493 |
| | Bacteroidia; Bacteroidales; | | | | |
| | Bacteroidaceae; | | | | |
| | Bacteroides; | | | | |
| r9_34597 | Bacteria; Firmicutes; | 0.002629844 | 0.64250637 | 0.041652036 | 0.393330562 |
| | Clostridia; Clostridiales; | | | | |
| | Eubacteriaceae; | | | | |
| | Eubacterium; | | | | |
| | [Eubacterium] eligens | | | | |
| r9_34599 | Bacteria; Firmicutes; | 2.34E−05 | 0.361707616 | 2.03E−08 | 0.577468192 |
| | Clostridia; Clostridiales;;; | | | | |
| r9_3478 | Bacteria; Firmicutes; | 0.049327894 | 0.30616915 | 0.007718992 | 0.441177736 |
| | Clostridia; Clostridiales; | | | | |
| | Ruminococcaceae;; | | | | |
| r9_34813 | Bacteria; Firmicutes; | 0.000367483 | −0.18517708 | 0.011560109 | 0.365573703 |
| | Clostridia; Clostridiales; | | | | |
| | Lachnospiraceae; | | | | |
| | Blautia; Blautia stercoris | | | | |
| r9_3482 | Bacteria; Firmicutes; | 0.028218102 | 0.454642443 | 0.000270538 | 0.230274493 |
| | Clostridia; Clostridiales; | | | | |
| | Lachnospiraceae;; | | | | |
| r9_3530 | Bacteria; Firmicutes; | 0.00783623 | 0.422794789 | 0.048309292 | 0.190511642 |
| | Clostridia; Clostridiates;;; | | | | |
| r9_354879 | * * * | 0.009839089 | 0.622037342 | 0.032319771 | −0.29568659 |
| r9_3606 | Bacteria; Firmicutes; | 1.12E−05 | −0.244095568 | 0.004107169 | −0.581499405 |
| | Clostridia; Clostridiales; | | | | |
| | Lachnospiraceae; | | | | |
| | Coprococcus; | | | | |
| r9_3608 | Bacteria; Firmicutes; | 0.001132639 | 0.474200454 | 4.48E−09 | 1.123064879 |
| | Clostridia; Clostridiales; | | | | |
| | Ruminococcaceae;; | | | | |
| r9_36184 | Bacteria; Firmicutes; | 0.009758349 | 0.199750911 | 0.000501794 | 0.66017138 |
| | Clostridia; Clostridiales;;; | | | | |
| r9_36192 | Bacteria; Firmicutes; | 0.004012927 | 0.530654618 | 0.000270571 | 0.886187409 |
| | Clostridia; Clostridiales; | | | | |
| | Lachnospiraceae; | | | | |
| | Blautia; Blautia sp. | | | | |
| | Marseille-P3201T | | | | |
| r9_362297 | Bacteria;;;;;; | 0.002326966 | 0.982692772 | 0.017709745 | 0.366955199 |
| r9_36269 | Bacteria; Bacteroidetes; | 0.033242551 | −0.065321844 | 2.42E−06 | 0.750003548 |
| | Bacteroidia; Bacteroidales; | | | | |
| | Bacteroidaceae;; | | | | |
| r9_363292 | Bacteria; Bacteroidetes; | 0.017646627 | −1.121719804 | 0.000228543 | −0.766431686 |
| | Bacteroidia; Bacteroidales; | | | | |
| | Bacteroidaceae; | | | | |
| | Bacteroides; uncultured | | | | |
| | Bacteroides sp. | | | | |
| r9_3634 | Bacteria; Firmicutes; | 5.86E−15 | −0.262764239 | 4.79E−06 | −0.329056276 |
| | Clostridia; Clostridiales; | | | | |
| | Lachnospiraceae;; | | | | |
| r9_36521 | Bacteria; Firmicutes; | 0.006468417 | 0.327038534 | 0.0120532.96 | 0.395462877 |
| | Clostridia; Clostridiales; | | | | |
| | Rumingcoccaceae; | | | | |
| | Faecalibacterium; | | | | |
| r9_3661 | Bacteria; Firmicutes; | 0.048735869 | −0.9572077 | 0.010496623 | −3.60466908 |
| | Clostridia; Clostridiales; | | | | |
| | Lachnospiraceae;; | | | | |
| r9_3664 | Bacteria; Firmicutes; | 0.030264428 | −0.080992838 | 2.52E−07 | 0.466230323 |
| | Clostridia; Clostridiales; | | | | |
| | Lachnospiraceae;; | | | | |
| r9_36987 | Bacteria; Firmicutes; | 0.001304277 | 0.23423138 | 1.96E−07 | 0.378108345 |
| | Clostridia; Clostridiales; | | | | |
| | Clostridiaceae;; | | | | |
| r9_371543 | | 0.049877137 | 1.561188675 | 0.000373494 | −3.98571212 |
| r9_37432 | Bacteria; Firmicutes; | 0.025967347 | 0.130262236 | 0.000920613 | 0.671443846 |
| | Clostridia; Clostridiales; | | | | |
| | Lachnospiraceae;; | | | | |
| r9_376190 | * * * | 0.008203302 | 1.366912418 | 0.031742547 | −2.174511365 |
| r9_377029 | Bacteria;;;;;; | 0.005315422 | 0.455840076 | 8.16E−05 | 0.499413691 |
| r9_378837 | Bacteria; Firmicutes; | 0.001170689 | 0.52978894 | 0.005435898 | 0.489596218 |
| | Clostridia; Clostridiates;;; | | | | |
| r9_3831 | Bacteria; Bacteroidetes; | 0.01543254 | 0.200217386 | 6.58E−09 | 1.505955006 |
| | Bacteroidia; Bacteroidales; | | | | |
| | Prevotellaceae; Prevotella; | | | | |
| r9_387436 | Bacteria; Firmicutes; | 0.045872965 | 0.496734167 | 0.001598197 | 0.591808507 |
| | Clostridia; Clostridiales; | | | | |
| | Clostridiaceae; | | | | |
| | Clostridium; Clostridium | | | | |
| | sp. AT4 | | | | |
| r9_38751 | Bacteria; Firmicules; | 1.81E−08 | −0.574665814 | 0.000717517 | −0.469394139 |
| | Clostridia; Clostridiales; | | | | |
| | Ruminococcaceae; | | | | |
| | Faecalibacterium; | | | | |
| r9_3876 | Bacteria; Firmicutes; | 0.003481792 | −0.138016432 | 0.001025426 | 0.501403495 |
| | Clostridia; Clostridiales;;; | | | | |
| r9_38857 | Bacteria; Firmicutes; | 0.000962838 | 0.273258656 | 0.005576409 | 0.328949461 |
| | Clostridia; Closindiales; | | | | |
| | Lachnospiraceae;; | | | | |
| r9_389 | * * * | 0.038792143 | 1.556621351 | 0.030429431 | −2.499867345 |
| r9_39132 | Bacteria; Bacteroldetes; | 0.00789985 | 0.57933885 | 6.88E−05 | 0.736701364 |
| | Bacteroidia; Bacteroidales; | | | | |
| | Bacteroidaceae; | | | | |
| | Bacteroides; | | | | |
| r9_394100 | Bacteria;;;;;; | 0.008870045 | 1.262895971 | 0.01856106 | 0.514392614 |
| r9_3975 | Bacteria; Firmicutes; | 4.27E−06 | −0.365064452 | 0.001531528 | −0.57386522 |
| | Clostridia; Clostridiales; | | | | |
| | Lachnospiraceae;; | | | | |
| r9_400891 | Bacteria; Firmicutes; | 0.015461762 | 0.729792335 | 0.039257903 | 0.427375651 |
| | Clostridia; Clostridiales; | | | | |
| | Lachnospiraceae; | | | | |
| | Blautia; Blautia stercoris | | | | |
| r9_40394 | Bacteria; Firmicutes; | 0.000989275 | −0.365227256 | 0.003726126 | −0.483370726 |
| | Clostridia; Clostridiales; | | | | |
| | Ruminococcaceae; | | | | |
| | Faecalibacterium; | | | | |
| r9_417377 | Bacteria; Firmicutes; | 0.020892026 | 0.855458538 | 0.02375474 | 0.59608451 |
| | Negativicutes; | | | | |
| | Veillonellales; | | | | |
| | Veillonellaceae; | | | | |
| | Megasphaera; | | | | |
| r9_42355 | Bacteria; Firmicutes; | 0.024460718 | 0.526476913 | 0.00765025 | 0.401427029 |
| | Clostridia; Clostridiales; | | | | |
| | Eubacteriaceae;; | | | | |
| r9_425510 | Bacteria; Bacteroidetes; | 0.031614282 | 0.451559623 | 0.029878926 | 2.238532727 |
| | Bacteroldia; Bacteroidales; | | | | |
| | Bacteroidaceae; | | | | |
| | Bacteroides; uncultured | | | | |
| | Bacteroides sp. | | | | |
| r9_4266 | Bacteria; Firmicutes; | 0.00078825 | −0.779761764 | 0.006234933 | −0.753652263 |
| | Clostridia; Clostridiales; | | | | |
| | Ruminococcaceae;; | | | | |
| r9_42723 | Bacteria; Firmicutes; | 0.006771337 | 0.257381036 | 0.019370549 | 1.126308393 |
| | Clostridia; Clostridiales; | | | | |
| | Clostridiaceae;; | | | | |
| r9_42962 | Bacteria; Firmicutes; | 0.005268165 | 0.269442531 | 1.57E−10 | 0.655608369 |
| | Clostridia; Clostridiales; | | | | |
| | Lachnospiraceae; | | | | |
| | Anaerotignum; | | | | |
| | Anaerotignum aminivorans | | | | |
| r9_43449 | Bacteria; Firmicutes; | 0.002108191 | 0.33777851 | 4.32E−06 | 0.74699265 |
| | Clostridia; Clostridiales; | | | | |
| | Lachnospiraceae; Blaulia; | | | | |
| r9_438 | Bacteria, Firmicutes;;;;; | 0.007989258 | −0.10616531 | 0.000802391 | 0.218605182 |
| r9_44237 | Bacteria; Firmicutes; | 0.000824319 | 0.730568645 | 0.006734949 | 0.39753584 |
| | Clostridia; Clostridiales; | | | | |
| | Lachnospiraceae; | | | | |
| | Roseburia; | | | | |
| | Roseburia intestinalis | | | | |
| r9_44463 | Bacteria; Firmicutes; | 0.000270305 | 0.213796602 | 0.033121804 | 0.298986614 |
| | Erysipelotrichia; | | | | |
| | Erysipelotrichales; | | | | |
| | Erysipelotrichaceas;; | | | | |
| r9_44503 | Bacteria; Firmicutes; | 0.01740993 | 0.19163378 | 2.97E−07 | 0.917179237 |
| | Clostridia; Clostnidiales; | | | | |
| | Lachnospiraceae;; | | | | |
| r9_44510 | Bacteria; Actinobacteria; | 0.00239369 | −0.127922874 | 0.014369151 | 0.436553873 |
| | Actinobacteria; | | | | |
| | Bifidobacteriales; | | | | |
| | Bifidobacteriaceae; | | | | |
| | Bifidobacterium; | | | | |
| r9_461 | Bacteria; Firmicules; | 8.95E−06 | −0.25007086 | 0.006057215 | −0.320892759 |
| | Clostridia; Clostridiales; | | | | |
| | Ruminococcaceae;; | | | | |
| r9_47647 | Bacteria; Firmicutes; | 0.005228167 | 0.8868288 | 0.002332709 | 0.72945884 |
| | Clostridia; Clostridiales; | | | | |
| | Clostridiaceae; | | | | |
| | Clostridium; uncultured | | | | |
| | Clostridium sp. | | | | |
| r9_47833 | Eukaryota; Nematoda; | 0.018909402 | 0.643982512 | 0.003174067 | 0.609754902 |
| | Enoplea; Trichinellida; | | | | |
| | Trichuridae; Trichuris; | | | | |
| | Trichuris trichiura | | | | |
| r9_5439 | Bacteria;;;;;; | 1.01E−07 | 0.258933478 | 2.33E−11 | 0.636216538 |
| r9_58174 | * * * | 0.04141856 | 0.440656789 | 0.002517797 | 0.326326359 |
| r9_58298 | Bacteria; Firmicutes; | 0.000483133 | 0.618355582 | 0.000117603 | 0.587622226 |
| | Clostridia; Clostridiales;;; | | | | |
| r9_58360 | Bacteria; Firmicutes; | 0.007731276 | 0.38626413 | 0.006997253 | 0.282851276 |
| | Clostridia; Clostridiales; | | | | |
| | Lachnospiraceae;; | | | | |
| r9_58388 | Bacteria; Firmicutes; | 0.036762328 | −0.377762486 | 0.020584076 | −0.432669681 |
| | Clostridia; Clostridiales; | | | | |
| | Lachnospiraceae;; | | | | |
| r9_58546 | Bacteria; Firmicutes: | 0.010321917 | 0.468241004 | 0.023019153 | 0.529237243 |
| | Clostridia; Clostridiales;;; | | | | |
| r9_5883 | Bacteria; Firmicutes; | 0.000580858 | 0.752062598 | 0.002434578 | 0.521257661 |
| | Clostridia; Clostridiales;;; | | | | |
| r9_591 | Bacteria; Firmicutes; | 0.000396856 i | −0.344197388 | 0.029997082 | −0.198190771 |
| | Clostridia; Clostridiales;;; | | | | |
| r9_59974 | Bacteria; Firmicutes; | 1.79E−05 | 0.418859102 | 0.001013621 | 0.319669987 |
| | Clostridia; Clostridiales;;; | | | | |
| r9_60 | Sacteria; Firmicutes; | 0.042274295 | 0.358936181 | 0.004972341 | 0.277467809 |
| | Clostridia; Clostridiales;;; | | | | |
| r9_6217 | Bacteria; Firmicutes; | 1.80E−07 | 0.196217888 | 0.033957826 | 0.315110339 |
| | Clostridia; Clostridiales; | | | | |
| | Lachnospiraceae; Blautia; | | | | |
| r9_6366 | Bacteria; Firmicutes; | 0.000195598 1 | 0.214074348 | 0.006807294 | 0.448251966 |
| | Clostridia; Clostridiales;;; | | | | |
| r9_63678 | Bacteria;;;;;; | 0.00199115 | 0.490543161 | 0.016943009 | 1.216360308 |
| r9_65609 | Bacteria; Firmicutes; | 0.001386044 | -0.38215756 | 3.11E−05 | −1.282133143 |
| | Bacilli; Bacillales;;; | | | | |
| r9_69177 | Bacteria; Firmicutes; | 3.05E−06 | 0.334371557 | 6.71E−06 | 0.605634928 |
| | Clostridia; Clostrdiales;;; | | | | |
| r9_73257 | | 0.007684842 | 1.005540162 | 0.005087604 | 0.533864117 |
| r9_733 | Bacteria; Firmicutes; | 0.004141611 | −0.138335398 | 0.000493783 | 0.295588039 |
| | Clostridia; Clostridiales;;; | | | | |
| r9_73382 | Bacteria; Firmicutes; | 0.045213108 | 0.464769678 | 0.000367216 | 0.843847156 |
| | Clostridia; Clostridiales; | | | | |
| | Lachnospiraceae; Blautia; | | | | |
| r9_73607 | Sacteria; Bacteroidetes; | 0.048713948 | −0.722317343 | 0.022674076 | −0.324076132 |
| | Bacteroidia; Bacteroidales; | | | | |
| | Bacteroidaceae;; | | | | |
| r9_73638 | Bacteria; Firmicutes; | 0.045530995 | 0.191280996 | 0.022251264 | 0.747850813 |
| | Clostridia; Clostridiales; | | | | |
| | Ruminococcacese; | | | | |
| | Ruminococcus; | | | | |
| | Rumingcoccus sp. WAL | | | | |
| | 17306 | | | | |
| r9_74469 | Bacteria; Firmicules; | 0.028871977 | 0.468102375 | 0.017992027 | 0.529478228 |
| | Clostridia; Clostridiales; | | | | |
| | Clostridiaceae;; | | | | |
| r9_74577 | Bacteria; Firmicutes; | 0.0117886 | 0.46251675 | 0.000935132 | −0.345740071 |
| | Clostridia; Clostridiales; | | | | |
| | Ruminococcaceae; | | | | |
| | Faecalibacterium; | | | | |
| r9_74893 | Bacteria; Firmicutes; | 0.046593622 | 0.613278114 | 0.016538623 | 0.72099247 |
| | Clostridia; Clostridiales; | | | | |
| | Ruminococcaceae; | | | | |
| | Faecalibacterium; | | | | |
| r9_77001 | Bacteria; Firmicutes; | 0.003948504 I | 0.53041472 | 1.67E−05 | 0.691383516 |
| | Clostridia; Clostridiales; | | | | |
| | Lachnospiraceae;; | | | | |
| r9_780 | Bacteria; Firmicutes; | 0.000593612 | 0.188856117 | 8.34E−06 | 0.495092627 |
| | Clostridia; Clostridiales; | | | | |
| | Ruminococcaceae;; | | | | |
| r9_79091 | Bacteria; Firmicutes; | 0.002812324 | 0.687357332 | 2.07E−09 | 2.238062464 |
| | Clostridia; Clostridiales; | | | | |
| | Ruminococcaceae; | | | | |
| | Fournierella; | | | | |
| | Fournierella massiliensis | | | | |
| r9_7978 | Bacteria; Bacteroidetes; | 0.045484427 | −0.198886102 | 0.000768145 | −0.874043894 |
| | Bacteroidia; | | | | |
| | Bacteroidales; | | | | |
| | Porphyromonadaceae; | | | | |
| | Porphyromonas; | | | | |
| r9_844 | Bacteria; Firmicules; | 4.54E−07 | 0.686628168 | 0.008048648 | 0.193593161 |
| | Clostridia; Clostridiales; | | | | |
| | Lachnospiraceae; Blautia; | | | | |
| | [Ruminococcus] gnavus | | | | |
| r9_850 | Bacteria; Firmicutes; | 0.01854747 | −0.11796444 | 0.007507546 | 0.348354822 |
| | Clostridia; Clostridiales; | | | | |
| | Lachnospiraceae; | | | | |
| | Lachnospira; Lachnospira | | | | |
| | pectinoschiza | | | | |
| r9_8764 | Bacteria; Firmicutes; | 0.00403717 | 0.152944954 | 0.022456313 | 0.635895057 |
| | Negativicutes; | | | | |
| | Veillonellales; | | | | |
| | Veillonellaceae; | | | | |
| | Megasphaera; | | | | |
| r9_88114 | Bacteria; Firmicutes; | 0.019340748 | 0.351153675 | 0.038898448 | 0.473720516 |
| | Clostridia; Clostridiales;;; | | | | |
| r9_917 | Bacteria; Firmicutes;;; | 0.002197994 | 0.493913689 | 2.71E−07 | 0.533617977 |
| r9_96656 | | 0.042809806 | 0.668016585 | 0.012379046 | −0.542188378 |
| r9_991 | Bacteria; Bacteroldetes; | 0.021177772 | 0.501525107 | 1.33E−07 | 1.112121952 |
| | Bacteroidia; Bacteroidales; | | | | |
| | Bacteroidaceae; | | | | |
| | Bacteroides; | | | | |
| | Bacteroides ovatus | | | | |

### Example 2: Specific Examples of Method for Detecting Taxa Co-Occurrence

In addition to the effect of gut microbiota by the antibiotic consumption in Example 1, the co-occurrence analysis was conducted in order to maintain, or add specific taxa by reflecting the association between several taxa, such as Genus level.

Additionally, as the gut microbiota is structured as a biological community, it is expected that most of the taxa will show negative and positive interactions with others. Knowing the interactions between different taxa gives us more options to preserve or re-introduce some depleted taxa into the gut community. For example, if we are interested in taxon A, but it is not possible to add it to a probiotic, we can instead add to the mix a different taxon B, which has a strong co-occurrence probability with taxon A.

Thus, the inventors performed a co-occurrence analysis in a subset of 100 users who did not consume antibiotics to find out which of the microorganisms inhabiting the gut have high probabilities of co-occurrence. In addition, the same co-occurrence analysis was performed for a subset of 100 users who did consume antibiotics. The tested each subset of 100 users was randomly extracted from the antibiotic consuming group and the antibiotic non-consuming group in Example 1.

A threshold of 0.85 was set as the minimum probability of co-occurrence useful for the purposes of this example, but any suitable threshold level can be set. The lists of co-occurring taxa at genus level are shown in table 2 in samples from antibiotic consumers, and table 3 in samples from antibiotic non-consumers. All analyses were conducted in R statistical software. Cooccur package was used for the co-occurrence analysis. However, any suitable statistical software and/or approaches and/or transformation software and/or approaches can be used.

That is, the probability of co-occurrence of genus in samples from antibiotic consumers is shown in Table 2, and the probability of co-occurrence of genus in samples from antibiotic non-consumers is shown in Table 3. In Tables 2 and 3, the column “prob_cooccur” represents the probability of finding the two organisms in the sample, the column “p_gt” represents the probability that when one of the taxa is present, the other is also present. The “effects” column represents the effect size of the association between the taxa.

TABLE 2
Probability of co-occurrence of genus in samples from antibiotic consumers

| sp1 | sp2 | sp1_inc | sp2_inc | obs_cooccur | prob_cooccur | exp_cooccur | p_lt | p_gt | sp1_name | sp2_name | effects |
|---|---|---|---|---|---|---|---|---|---|---|---|
| 95 | 110 | 100 | 100 | 100 | 1 | 100 | 1 | 1 | Bacteroides | Blautia | 0 |
| 95 | 489 | 100 | 100 | 100 | 1 | 100 | 1 | 1 | Bacteroides | Pseudo- | 0 |
| | | | | | | | | | | butyrivibrio | |
| 110 | 489 | 100 | 100 | 100 | 1 | 100 | 1 | 1 | Blautia | Pseudo- | 0 |
| | | | | | | | | | | butyrivibrio | |
| 95 | 213 | 100 | 99 | 99 | 0.99 | 99 | 1 | 1 | Bacteroides | Dorea | 0 |
| 95 | 584 | 100 | 99 | 99 | 0.99 | 99 | 1 | 1 | Bacteroides | Streptococcus | 0 |
| 110 | 213 | 100 | 99 | 99 | 0.99 | 99 | 1 | 1 | Blautia | Dorea | 0 |
| 110 | 584 | 100 | 99 | 99 | 0.99 | 99 | 1 | 1 | Blautia | Streptococcus | 0 |
| 213 | 489 | 99 | 100 | 99 | 0.99 | 99 | 1 | 1 | Dorea | Pseudo- | 0 |
| | | | | | | | | | | butyrivibrio | |
| 489 | 584 | 100 | 99 | 99 | 0.99 | 99 | 1 | 1 | Pseudo- | Streptococcus | 0 |
| | | | | | | | | | butyrivibrio | | |
| 95 | 254 | 100 | 98 | 98 | 0.98 | 98 | 1 | 1 | Bacteroides | Fusicatenibacter | 0 |
| 95 | 526 | 100 | 98 | 98 | 0.98 | 98 | 1 | 1 | Bacteroides | Roseburia | 0 |
| 95 | 586 | 100 | 98 | 98 | 0.98 | 98 | 1 | 1 | Bacteroides | Subdoligranulum | 0 |
| 110 | 254 | 100 | 98 | 98 | 0.98 | 98 | 1 | 1 | Blautia | Fusicatenibacter | 0 |
| 110 | 526 | 100 | 98 | 98 | 0.98 | 98 | 1 | 1 | Blautia | Roseburia | 0 |
| 110 | 586 | 100 | 98 | 98 | 0.98 | 98 | 1 | 1 | Blautia | Subdoligranulum | 0 |
| 213 | 584 | 99 | 99 | 98 | 0.98 | 98 | 0.99 | 1 | Dorea | Streptococcus | 0 |
| 254 | 489 | 98 | 100 | 98 | 0.98 | 98 | 1 | 1 | Fusicatenibacter | Pseudo- | 0 |
| | | | | | | | | | | butyrivibrio | |
| 489 | 526 | 100 | 98 | 98 | 0.98 | 98 | 1 | 1 | Pseudo- | Roseburia | 0 |
| | | | | | | | | | butyrivibrio | | |
| 489 | 586 | 100 | 98 | 98 | 0.98 | 98 | 1 | 1 | Pseudo- | Subdoligranulum | 0 |
| | | | | | | | | | butyrivibrio | | |
| 68 | 95 | 97 | 100 | 97 | 0.97 | 97 | 1 | 1 | Anaerostipes | Bacteroides | 0 |
| 68 | 110 | 97 | 100 | 97 | 0.97 | 97 | 1 | 1 | Anaerostipes | Blautia | 0 |
| 68 | 489 | 97 | 100 | 97 | 0.97 | 97 | 1 | 1 | Anaerostipes | Pseudo- | 0 |
| | | | | | | | | | | butyrivibrio | |
| 95 | 167 | 100 | 97 | 97 | 0.97 | 97 | 1 | 1 | Bacteroides | Clostridium | 0 |
| 110 | 167 | 100 | 97 | 97 | 0.97 | 97 | 1 | 1 | Blautia | Clostridium | 0 |
| 167 | 489 | 97 | 100 | 97 | 0.97 | 97 | 1 | 1 | Clostridium | Pseudo- | 0 |
| | | | | | | | | | | butyrivibrio | |
| 213 | 254 | 99 | 98 | 97 | 0.97 | 97 | 0.98 | 1 | Dorea | Fusicatenibacter | 0 |
| 254 | 584 | 98 | 99 | 97 | 0.97 | 97 | 0.98 | 1 | Fusicatenibacter | Streptococcus | 0 |
| 526 | 584 | 98 | 99 | 97 | 0.97 | 97 | 0.98 | 1 | Roseburia | Streptococcus | 0 |
| 584 | 586 | 99 | 98 | 97 | 0.97 | 97 | 0.98 | 1 | Streptococcus | Subdoligranulum | 0 |
| 213 | 526 | 99 | 98 | 98 | 0.97 | 97 | 1 | 0.02 | Dorea | Roseburia | 0.01 |
| 213 | 586 | 99 | 98 | 98 | 0.97 | 97 | 1 | 0.02 | Dorea | Subdoligranulum | 0.01 |
| 68 | 584 | 97 | 99 | 96 | 0.96 | 96 | 0.97 | 1 | Anaerostipes | Streptococcus | 0 |
| 95 | 169 | 100 | 96 | 96 | 0.96 | 96 | 1 | 1 | Bacteroides | Collinsella | 0 |
| 95 | 247 | 100 | 96 | 96 | 0.96 | 96 | 1 | 1 | Bacteroides | Flavonifractor | 0 |
| 110 | 169 | 100 | 96 | 96 | 0.96 | 96 | 1 | 1 | Blautia | Collinsella | 0 |
| 110 | 247 | 100 | 96 | 96 | 0.96 | 96 | 1 | 1 | Blautia | Flavonifractor | 0 |
| 167 | 584 | 97 | 99 | 96 | 0.96 | 96 | 0.97 | 1 | Clostridium | Streptococcus | 0 |
| 169 | 489 | 96 | 100 | 96 | 0.96 | 96 | 1 | 1 | Collinsella | Pseudo- | 0 |
| | | | | | | | | | | butyrivibrio | |
| 247 | 489 | 96 | 100 | 96 | 0.96 | 96 | 1 | 1 | Flavonifractor | Pseudo- | 0 |
| | | | | | | | | | | butyrivibrio | |
| 254 | 526 | 98 | 98 | 96 | 0.96 | 96 | 0.9602 | 1 | Fusicatenibacter | Roseburia | 0 |
| 254 | 586 | 98 | 98 | 96 | 0.96 | 96 | 0.9602 | 1 | Fusicatenibacter | Subdoligranulum | 0 |
| 526 | 586 | 98 | 98 | 97 | 0.96 | 96 | 0.9998 | 0.0398 | Roseburia | Subdoiigranulum | 0.01 |
| 68 | 213 | 97 | 99 | 97 | 0.96 | 96 | 1 | 0.03 | Anaerostipes | Dorea | 0.01 |
| 167 | 213 | 97 | 99 | 97 | 0.96 | 96 | 1 | 0.03 | Clostridium | Dorea | 0.01 |
| 68 | 254 | 97 | 98 | 95 | 0.951 | 95.1 | 0.94061 | 1 | Anaerostipes | Fusicatenibacter | −0.001 |
| 167 | 254 | 97 | 98 | 95 | 0.951 | 95.1 | 0.94061 | 1 | Clostridium | Fusicatenibacter | −0.001 |
| 68 | 586 | 97 | 98 | 96 | 0.951 | 95.1 | 0.99939 | 0.05939 | Anaerostipes | Subdoligranulum | 0.009 |
| 167 | 586 | 97 | 98 | 96 | 0.951 | 95.1 | 0.99939 | 0.05939 | Clostridium | Subdoligranulum | 0.009 |
| 68 | 526 | 97 | 98 | 97 | 0.951 | 95.1 | 1 | 0.00061 | Anaerostipes | Roseburia | 0.019 |
| 167 | 526 | 97 | 98 | 97 | 0.951 | 95.1 | 1 | 0.00061 | Clostridium | Roseburia | 0.019 |
| 95 | 237 | 100 | 95 | 95 | 0.95 | 95 | 1 | 1 | Bacteroides | Faecalibacterium | 0 |
| 95 | 302 | 100 | 95 | 95 | 0.95 | 95 | 1 | 1 | Bacteroides | Intestinibacter | 0 |
| 110 | 237 | 100 | 95 | 95 | 0.95 | 95 | 1 | 1 | Blautia | Faecalibacterium | 0 |
| 110 | 302 | 100 | 95 | 95 | 0.95 | 95 | 1 | 1 | Blautia | Intestinibacter | 0 |
| 169 | 584 | 96 | 99 | 95 | 0.95 | 95 | 0.96 | 1 | Collinsella | Streptococcus | 0 |
| 213 | 247 | 99 | 96 | 95 | 0.95 | 95 | 0.96 | 1 | Dorea | Flavonifractor | 0 |
| 237 | 489 | 95 | 100 | 95 | 0.95 | 95 | 1 | 1 | Faecalibacterium | Pseudo- | 0 |
| | | | | | | | | | | butyrivibrio | |
| 247 | 584 | 96 | 99 | 95 | 0.95 | 95 | 0.96 | 1 | Flavonifractor | Streptococcus | 0 |
| 302 | 489 | 95 | 100 | 95 | 0.95 | 95 | 1 | 1 | Intestinibacter | Pseudo- | 0 |
| | | | | | | | | | | butyrivibrio | |
| 169 | 213 | 96 | 99 | 96 | 0.95 | 95 | 1 | 0.04 | Collinsella | Dorea | 0.01 |
| 169 | 254 | 96 | 98 | 94 | 0.941 | 94.1 | 0.92121 | 1 | Collinsella | Fusicatenibacter | −0.001 |
| 247 | 254 | 96 | 98 | 94 | 0.941 | 94.1 | 0.92121 | 1 | Flavonifractor | Fusicatenibacter | −0.001 |
| 247 | 526 | 96 | 98 | 94 | 0.941 | 94.1 | 0.92121 | 1 | Flavonifractor | Roseburia | −0.001 |
| 247 | 586 | 96 | 98 | 95 | 0.941 | 94.1 | 0.99879 | 0.07879 | Flavonifractor | Subdoligranulum | 0.009 |
| 68 | 167 | 97 | 97 | 96 | 0.941 | 94.1 | 0.99999 | 0.00181 | Anaerostipes | Clostridium | 0.019 |
| 169 | 526 | 96 | 98 | 96 | 0.941 | 94.1 | 1 | 0.00121 | Collinsella | Roseburia | 0.019 |
| 169 | 586 | 96 | 98 | 96 | 0.941 | 94.1 | 1 | 0.00121 | Collinsella | Subdoligranulum | 0.019 |
| 95 | 539 | 100 | 94 | 94 | 0.94 | 94 | 1 | 1 | Bacteroides | Sarcina | 0 |
| 110 | 539 | 100 | 94 | 94 | 0.94 | 94 | 1 | 1 | Blautia | Sarcina | 0 |
| 213 | 237 | 99 | 95 | 94 | 0.94 | 94 | 0.95 | 1 | Dorea | Faecalibacterium | 0 |
| 237 | 584 | 95 | 99 | 94 | 0.94 | 94 | 0.95 | 1 | Faecalibacterium | Streptococcus | 0 |
| 302 | 584 | 95 | 99 | 94 | 0.94 | 94 | 0.95 | 1 | Intestinibacter | Streptococcus | 0 |
| 489 | 539 | 100 | 94 | 94 | 0.94 | 94 | 1 | 1 | Pseudo- | Sarcina | 0 |
| | | | | | | | | | butyrivibrio | | |
| 213 | 302 | 99 | 95 | 95 | 0.94 | 94 | 1 | 0.05 | Dorea | Intestinibacter | 0.01 |
| 68 | 247 | 97 | 96 | 93 | 0.931 | 93.1 | 0.88361 | 1 | Anaerostipes | Flavonifractor | −0.001 |
| 213 | 539 | 99 | 94 | 93 | 0.931 | 93.1 | 0.94 | 1 | Dorea | Sarcina | −0.001 |
| 237 | 254 | 95 | 98 | 93 | 0.931 | 93.1 | 0.90202 | 1 | Faecalibacterium | Fusicatenibacter | −0.001 |
| 237 | 526 | 95 | 98 | 93 | 0.931 | 93.1 | 0.90202 | 1 | Faecalibacterium | Roseburia | −0.001 |
| 237 | 586 | 95 | 98 | 93 | 0.931 | 93.1 | 0.90202 | 1 | Faecalibacterium | Subdoligranulum | −0.001 |
| 539 | 584 | 94 | 99 | 93 | 0.931 | 93.1 | 0.94 | 1 | Sarcina | Streptococcus | −0.001 |
| 167 | 247 | 97 | 96 | 94 | 0.931 | 93.1 | 0.99641 | 0.11639 | Clostridium | Flavonifractor | 0.009 |
| 254 | 302 | 98 | 95 | 94 | 0.931 | 93.1 | 0.99798 | 0.09798 | Fusicatenibacter | Intestinibacter | 0.009 |
| 302 | 526 | 95 | 98 | 94 | 0.931 | 93.1 | 0.99798 | 0.09798 | Intestinibacter | Roseburia | 0.009 |
| 302 | 586 | 95 | 98 | 94 | 0.931 | 93.1 | 0.99798 | 0.09798 | Intestinibacter | Subdoligranulum | 0.009 |
| 68 | 169 | 97 | 96 | 95 | 0.931 | 93.1 | 0.99998 | 0.00359 | Anaerostipes | Collinsella | 0.019 |
| 167 | 169 | 97 | 96 | 95 | 0.931 | 93.1 | 0.99998 | 0.00359 | Clostridium | Collinsella | 0.019 |
| 42 | 95 | 93 | 100 | 93 | 0.93 | 93 | 1 | 1 | Alistipes | Bacteroides | 0 |
| 42 | 110 | 93 | 100 | 93 | 0.93 | 93 | 1 | 1 | Alistipes | Blautia | 0 |
| 42 | 489 | 93 | 100 | 93 | 0.93 | 93 | 1 | 1 | Alistipes | Pseudo- | 0 |
| | | | | | | | | | | butyrivibrio | |
| 167 | 237 | 97 | 95 | 92 | 0.922 | 92.2 | 0.856 | 1 | Clostridium | Faecalibacterium | −0.002 |
| 169 | 247 | 96 | 96 | 93 | 0.922 | 92.2 | 0.99292 | 0.15283 | Collinsella | Flavonifractor | 0.008 |
| 68 | 237 | 97 | 95 | 93 | 0.922 | 92.2 | 0.99406 | 0.144 | Anaerostipes | Faecalibacterium | 0.008 |
| 167 | 302 | 97 | 95 | 93 | 0.922 | 92.2 | 0.99406 | 0.144 | Clostridium | Intestinibacter | 0.008 |
| 68 | 302 | 97 | 95 | 94 | 0.922 | 92.2 | 0.99994 | 0.00594 | Anaerostipes | Intestinibacter | 0.018 |
| 42 | 213 | 93 | 99 | 92 | 0.921 | 92.1 | 0.93 | 1 | Alistipes | Dorea | −0.001 |
| 42 | 584 | 93 | 99 | 92 | 0.921 | 92.1 | 0.93 | 1 | Alistipes | Streptococcus | −0.001 |
| 254 | 539 | 98 | 94 | 92 | 0.921 | 92.1 | 0.88303 | 1 | Fusicatenibacter | Sarcina | −0.001 |
| 526 | 539 | 98 | 94 | 92 | 0.921 | 92.1 | 0.88303 | 1 | Roseburia | Sarcina | −0.001 |
| 539 | 586 | 94 | 98 | 92 | 0.921 | 92.1 | 0.88303 | 1 | Sarcina | Subdoligranulum | −0.001 |
| 95 | 231 | 100 | 92 | 92 | 0.92 | 92 | 1 | 1 | Bacteroides | Erysipelatoclos- | 0 |
| | | | | | | | | | | tridium | |
| 110 | 231 | 100 | 92 | 92 | 0.92 | 92 | 1 | 1 | Blautia | Erysipelatoclos- | 0 |
| | | | | | | | | | | tridium | |
| 231 | 489 | 92 | 100 | 92 | 0.92 | 92 | 1 | 1 | Erysipelatoclos- | Pseudo- | 0 |
| | | | | | | | | | tridium | butyrivibrio | |
| 169 | 237 | 96 | 95 | 91 | 0.912 | 91.2 | 0.81188 | 1 | Collinsella | Faecalibacterium | −0.002 |
| 237 | 247 | 95 | 96 | 91 | 0.912 | 91.2 | 0.81188 | 1 | Faecalibacterium | Flavonifractor | −0.002 |
| 247 | 302 | 96 | 95 | 91 | 0.912 | 91.2 | 0.81188 | 1 | Flavonifractor | Intestinibacter | −0.002 |
| 169 | 302 | 96 | 95 | 92 | 0.912 | 91.2 | 0.98837 | 0.18812 | Collinsella | Intestinibacter | 0.008 |
| 68 | 539 | 97 | 94 | 92 | 0.912 | 91.2 | 0.99116 | 0.17103 | Anaerostipes | Sarcina | 0.008 |
| 167 | 539 | 97 | 94 | 92 | 0.912 | 91.2 | 0.99116 | 0.17103 | Clostridium | Sarcina | 0.008 |
| 42 | 254 | 93 | 98 | 91 | 0.911 | 91.1 | 0.86424 | 1 | Alistipes | Fusicatenibacter | −0.001 |
| 42 | 526 | 93 | 98 | 91 | 0.911 | 91.1 | 0.86424 | 1 | Alistipes | Roseburia | −0.001 |
| 231 | 584 | 92 | 99 | 91 | 0.911 | 91.1 | 0.92 | 1 | Erysipeiatoclos- | Streptococcus | −0.001 |
| | | | | | | | | | tridium | | |
| 42 | 586 | 93 | 98 | 92 | 0.911 | 91.1 | 0.99576 | 0.13576 | Alistipes | Subdoligranulum | 0.009 |
| 213 | 231 | 99 | 92 | 92 | 0.911 | 91.1 | 1 | 0.08 | Dorea | Erysipelatoclos- | 0.009 |
| | | | | | | | | | | tridium | |
| 42 | 167 | 93 | 97 | 90 | 0.902 | 90.2 | 0.80251 | 1 | Alistipes | Clostridium | −0.002 |
| 169 | 539 | 96 | 94 | 90 | 0.902 | 90.2 | 0.77769 | 1 | Collinsella | Sarcina | −0.002 |
| 231 | 254 | 92 | 98 | 90 | 0.902 | 90.2 | 0.84566 | 1 | Erysipelatoclos- | Fusicatenibacter | −0.002 |
| | | | | | | | | | tridium | | |
| 237 | 302 | 95 | 95 | 91 | 0.902 | 90.2 | 0.98102 | 0.23041 | Faecalibacterium | Intestinibacter | 0.008 |
| 247 | 539 | 96 | 94 | 91 | 0.902 | 90.2 | 0.9828 | 0.22231 | Flavonifractor | Sarcina | 0.008 |
| 42 | 68 | 93 | 97 | 91 | 0.902 | 90.2 | 0.98771 | 0.19749 | Alistipes | Anaerostipes | 0.008 |
| 231 | 586 | 92 | 98 | 91 | 0.902 | 90.2 | 0.99434 | 0.15434 | Erysipelatoclos- | Subdoligranulum | 0.008 |
| | | | | | | | | | tridium | | |
| 231 | 526 | 92 | 98 | 92 | 0.902 | 90.2 | 1 | 0.00566 | Erysipelatoclos- | Roseburia | 0.018 |
| | | | | | | | | | tridium | | |
| 95 | 426 | 100 | 90 | 90 | 0.9 | 90 | 1 | 1 | Bacteroides | Parabacteroides | 0 |
| 110 | 426 | 100 | 90 | 90 | 0.9 | 90 | 1 | 1 | Blautia | Parabacteroides | 0 |
| 426 | 489 | 90 | 100 | 90 | 0.9 | 90 | 1 | 1 | Parabacteroides | Pseudo- | 0 |
| | | | | | | | | | | butyrivibrio | |
| 302 | 539 | 95 | 94 | 90 | 0.893 | 89.3 | 0.97211 | 0.27091 | Intestinibacter | Sarcina | 0.007 |
| 42 | 247 | 93 | 96 | 90 | 0.893 | 89.3 | 0.97625 | 0.2554 | Alistipes | Flavonifractor | 0.007 |
| 237 | 539 | 95 | 94 | 91 | 0.893 | 89.3 | 0.99882 | 0.02789 | Faecalibacterium | Sarcina | 0.017 |
| 42 | 169 | 93 | 96 | 91 | 0.893 | 89.3 | 0.99916 | 0.02375 | Alistipes | Collinsella | 0.017 |
| 167 | 231 | 97 | 92 | 91 | 0.892 | 89.2 | 0.99965 | 0.01628 | Clostridium | Erysipeiatoclos- | 0.018 |
| | | | | | | | | | | tridium | |
| 68 | 231 | 97 | 92 | 92 | 0.892 | 89.2 | 1 | 0.00035 | Anaerostipes | Erysipelatoclos- | 0.028 |
| | | | | | | | | | | tridium | |
| 213 | 426 | 99 | 90 | 89 | 0.891 | 89.1 | 0.9 | 1 | Dorea | Parabacteroides | −0.001 |
| 426 | 584 | 90 | 99 | 89 | 0.891 | 89.1 | 0.9 | 1 | Parabacteroides | Streptococcus | −0.001 |
| 42 | 302 | 93 | 95 | 89 | 0.884 | 88.3 | 0.96177 | 0.3097 | Alistipes | Intestinibacter | 0.007 |
| 42 | 237 | 93 | 95 | 91 | 0.884 | 88.3 | 0.99996 | 0.00203 | Alistipes | Faecalibacterium | 0.027 |
| 231 | 247 | 92 | 96 | 88 | 0.883 | 88.3 | 0.71257 | 1 | Erysipelatoclos- | Flavonifractor | −0.003 |
| | | | | | | | | | tridium | | |
| 169 | 231 | 96 | 92 | 90 | 0.883 | 88.3 | 0.99867 | 0.03122 | Collinsella | Erysipelatodos- | 0.017 |
| | | | | | | | | | | tridium | |
| 254 | 426 | 98 | 90 | 88 | 0.882 | 88.2 | 0.80909 | 1 | Fusicatenibacter | Parabacteroides | −0.002 |
| 426 | 526 | 90 | 98 | 89 | 0.882 | 88.2 | 0.99091 | 0.19091 | Parabacteroides | Roseburia | 0.008 |
| 426 | 586 | 90 | 98 | 89 | 0.882 | 88.2 | 0.99091 | 0.19091 | Parabacteroides | Subdoligranulum | 0.008 |
| 69 | 95 | 88 | 100 | 88 | 0.88 | 88 | 1 | 1 | Anaerotruncus | Bacteroides | 0 |
| 69 | 110 | 88 | 100 | 88 | 0.88 | 88 | 1 | 1 | Anaerotruncus | Blautia | 0 |
| 69 | 489 | 88 | 100 | 88 | 0.88 | 88 | 1 | 1 | Anaerotruncus | Pseudo- | 0 |
| | | | | | | | | | | butyrivibrio | |
| 231 | 237 | 92 | 95 | 88 | 0.874 | 87.4 | 0.9501 | 0.34681 | Erysipelatodos- | Faecalibacterium | 0.006 |
| | | | | | | | | | tridium | | |
| 42 | 539 | 93 | 94 | 89 | 0.874 | 87.4 | 0.99606 | 0.05537 | Alistipes | Sarcina | 0.016 |
| 231 | 302 | 92 | 95 | 89 | 0.874 | 87.4 | 0.9968 | 0.0499 | Erysipelatoclos- | Intestinibacter | 0.016 |
| | | | | | | | | | tridium | | |
| 68 | 426 | 97 | 90 | 88 | 0.873 | 87.3 | 0.97421 | 0.27347 | Anaerostipes | Parabacteroides | 0.007 |
| 167 | 426 | 97 | 90 | 88 | 0.873 | 87.3 | 0.97421 | 0.27347 | Clostridium | Parabacteroides | 0.007 |
| 69 | 584 | 88 | 99 | 87 | 0.871 | 87.1 | 0.88 | 1 | Anaerotruncus | Streptococcus | −0.001 |
| 69 | 213 | 88 | 99 | 88 | 0.871 | 87.1 | 1 | 0.12 | Anaerotruncus | Dorea | 0.009 |
| 231 | 539 | 92 | 94 | 88 | 0.865 | 86.5 | 0.99385 | 0.07178 | Erysipelatoclos- | Sarcina | 0.015 |
| | | | | | | | | | tridium | | |
| 247 | 426 | 96 | 90 | 87 | 0.864 | 86.4 | 0.95123 | 0.34837 | Flavonifractor | Parabacteroides | 0.006 |
| 169 | 426 | 96 | 90 | 89 | 0.864 | 86.4 | 0.99995 | 0.00281 | Collinsella | Parabacteroides | 0.026 |
| 69 | 254 | 88 | 98 | 87 | 0.862 | 86.2 | 0.98667 | 0.22667 | Anaerotruncus | Fusicatenibacter | 0.008 |
| 69 | 526 | 88 | 98 | 87 | 0.862 | 86.2 | 0.98667 | 0.22667 | Anaerotruncus | Roseburia | 0.008 |
| 69 | 586 | 88 | 98 | 88 | 0.862 | 86.2 | 1 | 0.01333 | Anaerotruncus | Subdoligranulum | 0.018 |
| 42 | 231 | 93 | 92 | 86 | 0.856 | 85.6 | 0.90364 | 0.45272 | Alistipes | Erysipelatoclos- | 0.004 |
| | | | | | | | | | | tridium | |
| 237 | 426 | 95 | 90 | 86 | 0.855 | 85.5 | 0.92314 | 0.41625 | Faecalibacterium | Parabacteroides | 0.005 |
| 302 | 426 | 95 | 90 | 86 | 0.855 | 85.5 | 0.92314 | 0.41625 | Intestinibacter | Parabacteroides | 0.005 |
| 68 | 69 | 97 | 88 | 87 | 0.854 | 85.4 | 0.99864 | 0.03728 | Anaerostipes | Anaerotruncus | 0.016 |
| 69 | 167 | 88 | 97 | 87 | 0.854 | 85.4 | 0.99864 | 0.03728 | Anaerotruncus | Clostridium | 0.016 |

TABLE 3
Probability of co-occurrence of genus in samples from antibiotic non-consumers.

| sp1 | sp2 | sp1_inc | sp2_inc | obs_cooccur | prob_cooccur | exp_cooccur | p_lt | p_gt | sp1_name | sp2_name | effects |
|---|---|---|---|---|---|---|---|---|---|---|---|
| 68 | 95 | 100 | 100 | 100 | 1 | 100 | 1 | 1 | Anaerostipes | Bacteroides | 0 |
| 68 | 110 | 100 | 100 | 100 | 1 | 100 | 1 | 1 | Anaerostipes | Blautia | 0 |
| 68 | 167 | 100 | 100 | 100 | 1 | 100 | 1 | 1 | Anaerostipes | Clostridium | 0 |
| 68 | 213 | 100 | 100 | 100 | 1 | 100 | 1 | 1 | Anaerostipes | Dorea | 0 |
| 68 | 237 | 100 | 100 | 100 | 1 | 100 | 1 | 1 | Anaerostipes | Faecalibacterium | 0 |
| 68 | 247 | 100 | 100 | 100 | 1 | 100 | 1 | 1 | Anaerostipes | Flavonifractor | 0 |
| 68 | 489 | 100 | 100 | 100 | 1 | 100 | 1 | 1 | Anaerostipes | Pseudobutyrivibrio | 0 |
| 68 | 526 | 100 | 100 | 100 | 1 | 100 | 1 | 1 | Anaerostipes | Roseburia | 0 |
| 95 | 110 | 100 | 100 | 100 | 1 | 100 | 1 | 1 | Bacteroides | Blautia | 0 |
| 95 | 167 | 100 | 100 | 100 | 1 | 100 | 1 | 1 | Bacteroides | Clostridium | 0 |
| 95 | 213 | 100 | 100 | 100 | 1 | 100 | 1 | 1 | Bacteroides | Dorea | 0 |
| 95 | 237 | 100 | 100 | 100 | 1 | 100 | 1 | 1 | Bacteroides | Faecalibacterium | 0 |
| 95 | 247 | 100 | 100 | 100 | 1 | 100 | 1 | 1 | Bacteroides | Flavonifractor | 0 |
| 95 | 489 | 100 | 100 | 100 | 1 | 100 | 1 | 1 | Bacteroides | Pseudobutyrivibrio | 0 |
| 95 | 526 | 100 | 100 | 100 | 1 | 100 | 1 | 1 | Bacteroides | Roseburia | 0 |
| 110 | 167 | 100 | 100 | 100 | 1 | 100 | 1 | 1 | Blautia | Clostridium | 0 |
| 110 | 213 | 100 | 100 | 100 | 1 | 100 | 1 | 1 | Blautia | Dorea | 0 |
| 110 | 237 | 100 | 100 | 100 | 1 | 100 | 1 | 1 | Blautia | Faecalibacterium | 0 |
| 110 | 247 | 100 | 100 | 100 | 1 | 100 | 1 | 1 | Blautia | Flavonifractor | 0 |
| 110 | 489 | 100 | 100 | 100 | 1 | 100 | 1 | 1 | Blautia | Pseudobutyrivibrio | 0 |
| 110 | 526 | 100 | 100 | 100 | 1 | 100 | 1 | 1 | Blautia | Roseburia | 0 |
| 167 | 213 | 100 | 100 | 100 | 1 | 100 | 1 | 1 | Clostridium | Dorea | 0 |
| 167 | 237 | 100 | 100 | 100 | 1 | 100 | 1 | 1 | Clostridium | Faecalibacterium | 0 |
| 167 | 247 | 100 | 100 | 100 | 1 | 100 | 1 | 1 | Clostridium | Flavonifractor | 0 |
| 167 | 489 | 100 | 100 | 100 | 1 | 100 | 1 | 1 | Clostridium | Pseudobutyrivibrio | 0 |
| 167 | 526 | 100 | 100 | 100 | 1 | 100 | 1 | 1 | Clostridium | Roseburia | 0 |
| 213 | 237 | 100 | 100 | 100 | 1 | 100 | 1 | 1 | Dorea | Faecalibacterium | 0 |
| 213 | 247 | 100 | 100 | 100 | 1 | 100 | 1 | 1 | Dorea | Flavonifractor | 0 |
| 213 | 489 | 100 | 100 | 100 | 1 | 100 | 1 | 1 | Dorea | Pseudobutyrivibrio | 0 |
| 213 | 526 | 100 | 100 | 100 | 1 | 100 | 1 | 1 | Dorea | Roseburia | 0 |
| 237 | 247 | 100 | 100 | 100 | 1 | 100 | 1 | 1 | Faecalibacterium | Flavonifractor | 0 |
| 237 | 489 | 100 | 100 | 100 | 1 | 100 | 1 | 1 | Faecalibacterium | Pseudobutyrivibrio | 0 |
| 237 | 526 | 100 | 100 | 100 | 1 | 100 | 1 | 1 | Faecalibacterium | Roseburia | 0 |
| 247 | 489 | 100 | 100 | 100 | 1 | 100 | 1 | 1 | Flavonifractor | Pseudobutyrivibrio | 0 |
| 247 | 526 | 100 | 100 | 100 | 1 | 100 | 1 | 1 | Flavonifractor | Roseburia | 0 |
| 489 | 526 | 100 | 100 | 100 | 1 | 100 | 1 | 1 | Pseudobutyrivibrio | Roseburia | 0 |
| 68 | 169 | 100 | 99 | 99 | 0.99 | 99 | 1 | 1 | Anaerostipes | Collinsella | 0 |
| 68 | 231 | 100 | 99 | 99 | 0.99 | 99 | 1 | 1 | Anaerostipes | Erysipelatoclos- | 0 |
| | | | | | | | | | | tridium | |
| 68 | 539 | 100 | 99 | 99 | 0.99 | 99 | 1 | 1 | Anaerostipes | Sarcina | 0 |
| 95 | 169 | 100 | 99 | 99 | 0.99 | 99 | 1 | 1 | Bacteroides | Collinsella | 0 |
| 95 | 231 | 100 | 99 | 99 | 0.99 | 99 | 1 | 1 | Bacteroides | Erysipelatoclos- | 0 |
| | | | | | | | | | | tridium | |
| 95 | 539 | 100 | 99 | 99 | 0.99 | 99 | 1 | 1 | Bacteroides | Sarcina | 0 |
| 110 | 169 | 100 | 99 | 99 | 0.99 | 99 | 1 | 1 | Blautia | Collinsella | 0 |
| 110 | 231 | 100 | 99 | 99 | 0.99 | 99 | 1 | 1 | Blautia | Erysipelatoclos- | 0 |
| | | | | | | | | | | tridium | |
| 110 | 539 | 100 | 99 | 99 | 0.99 | 99 | 1 | 1 | Blautia | Sarcina | 0 |
| 167 | 169 | 100 | 99 | 99 | 0.99 | 99 | 1 | 1 | Clostridium | Collinsella | 0 |
| 167 | 231 | 100 | 99 | 99 | 0.99 | 99 | 1 | 1 | Clostridium | Erysipelatoclos- | 0 |
| | | | | | | | | | | tridium | |
| 167 | 539 | 100 | 99 | 99 | 0.99 | 99 | 1 | 1 | Clostridium | Sarcina | 0 |
| 169 | 213 | 99 | 100 | 99 | 0.99 | 99 | 1 | 1 | Collinsella | Dorea | 0 |
| 169 | 237 | 99 | 100 | 99 | 0.99 | 99 | 1 | 1 | Collinsella | Faecalibacterium | 0 |
| 169 | 247 | 99 | 100 | 99 | 0.99 | 99 | 1 | 1 | Collinsella | Flavonifractor | 0 |
| 169 | 489 | 99 | 100 | 99 | 0.99 | 99 | 1 | 1 | Collinsella | Pseudobutyrivibrio | 0 |
| 169 | 526 | 99 | 100 | 99 | 0.99 | 99 | 1 | 1 | Collinsella | Roseburia | 0 |
| 213 | 231 | 100 | 99 | 99 | 0.99 | 99 | 1 | 1 | Dorea | Erysipelatoclos- | 0 |
| | | | | | | | | | | tridium | |
| 213 | 539 | 100 | 99 | 99 | 0.99 | 99 | 1 | 1 | Dorea | Sarcina | 0 |
| 231 | 237 | 99 | 100 | 99 | 0.99 | 99 | 1 | 1 | Erysipelatoclos- | Faecalibacterium | 0 |
| | | | | | | | | | tridium | | |
| 231 | 247 | 99 | 100 | 99 | 0.99 | 99 | 1 | 1 | Erysipelatoclos- | Flavonifractor | 0 |
| | | | | | | | | | tridium | | |
| 231 | 489 | 99 | 100 | 99 | 0.99 | 99 | 1 | 1 | Erysipelatoclos- | Pseudobutyrivibrio | 0 |
| | | | | | | | | | tridium | | |
| 231 | 526 | 99 | 100 | 99 | 0.99 | 99 | 1 | 1 | Erysipelatoclos- | Roseburia | 0 |
| | | | | | | | | | tridium | | |
| 237 | 539 | 100 | 99 | 99 | 0.99 | 99 | 1 | 1 | Faecalibacterium | Sarcina | 0 |
| 247 | 539 | 100 | 99 | 99 | 0.99 | 99 | 1 | 1 | Flavonifractor | Sarcina | 0 |
| 489 | 539 | 100 | 99 | 99 | 0.99 | 99 | 1 | 1 | Pseudobutyrivibrio | Sarcina | 0 |
| 526 | 539 | 100 | 99 | 99 | 0.99 | 99 | 1 | 1 | Roseburia | Sarcina | 0 |
| 68 | 254 | 100 | 98 | 98 | 0.98 | 98 | 1 | 1 | Anaerostipes | Fusicatenibacter | 0 |
| 68 | 302 | 100 | 98 | 98 | 0.98 | 98 | 1 | 1 | Anaerostipes | Intestinibacter | 0 |
| 68 | 426 | 100 | 98 | 98 | 0.98 | 98 | 1 | 1 | Anaerostipes | Parabacteroides | 0 |
| 68 | 586 | 100 | 98 | 98 | 0.98 | 98 | 1 | 1 | Anaerostipes | Subdoligranulum | 0 |
| 95 | 254 | 100 | 98 | 98 | 0.98 | 98 | 1 | 1 | Bacteroides | Fusicatenibacter | 0 |
| 95 | 302 | 100 | 98 | 98 | 0.98 | 98 | 1 | 1 | Bacteroides | Intestinibacter | 0 |
| 95 | 426 | 100 | 98 | 98 | 0.98 | 98 | 1 | 1 | Bacteroides | Parabacteroides | 0 |
| 95 | 586 | 100 | 98 | 98 | 0.98 | 98 | 1 | 1 | Bacteroides | Subdoligranulum | 0 |
| 110 | 254 | 100 | 98 | 98 | 0.98 | 98 | 1 | 1 | Blautia | Fusicatenibacter | 0 |
| 110 | 302 | 100 | 98 | 98 | 0.98 | 98 | 1 | 1 | Blautia | Intestinibacter | 0 |
| 110 | 426 | 100 | 98 | 98 | 0.98 | 98 | 1 | 1 | Blautia | Parabacteroides | 0 |
| 110 | 586 | 100 | 98 | 98 | 0.98 | 98 | 1 | 1 | Blautia | Subdoligranulum | 0 |
| 167 | 254 | 100 | 98 | 98 | 0.98 | 98 | 1 | 1 | Clostridium | Fusicatenibacter | 0 |
| 167 | 302 | 100 | 98 | 98 | 0.98 | 98 | 1 | 1 | Clostridium | Intestinibacter | 0 |
| 167 | 426 | 100 | 98 | 98 | 0.98 | 98 | 1 | 1 | Clostridium | Parabacteroides | 0 |
| 167 | 586 | 100 | 98 | 98 | 0.98 | 98 | 1 | 1 | Clostridium | Subdoligranulum | 0 |
| 169 | 231 | 99 | 99 | 98 | 0.98 | 98 | 0.99 | 1 | Collinsella | Erysipelatoclos- | 0 |
| | | | | | | | | | | tridium | |
| 169 | 539 | 99 | 99 | 98 | 0.98 | 98 | 0.99 | 1 | Collinsella | Sarcina | 0 |
| 213 | 254 | 100 | 98 | 98 | 0.98 | 98 | 1 | 1 | Dorea | Fusicatenibacter | 0 |
| 213 | 302 | 100 | 98 | 98 | 0.98 | 98 | 1 | 1 | Dorea | Intestinibacter | 0 |
| 213 | 426 | 100 | 98 | 98 | 0.98 | 98 | 1 | 1 | Dorea | Parabacteroides | 0 |
| 213 | 586 | 100 | 98 | 98 | 0.98 | 98 | 1 | 1 | Dorea | Subdoligranulum | 0 |
| 231 | 539 | 99 | 99 | 98 | 0.98 | 98 | 0.99 | 1 | Erysipelatoclos- | Sarcina | 0 |
| | | | | | | | | | tridium | | |
| 237 | 254 | 100 | 98 | 98 | 0.98 | 98 | 1 | 1 | Faecalibacterium | Fusicatenibacter | 0 |
| 237 | 302 | 100 | 98 | 98 | 0.98 | 98 | 1 | 1 | Faecalibacterium | Intestinibacter | 0 |
| 237 | 426 | 100 | 98 | 98 | 0.98 | 98 | 1 | 1 | Faecalibacterium | Parabacteroides | 0 |
| 237 | 586 | 100 | 98 | 98 | 0.98 | 98 | 1 | 1 | Faecalibacterium | Subdoligranulum | 0 |
| 247 | 254 | 100 | 98 | 98 | 0.98 | 98 | 1 | 1 | Flavonifractor | Fusicatenibacter | 0 |
| 247 | 302 | 100 | 98 | 98 | 0.98 | 98 | 1 | 1 | Flavonifractor | Intestinibacter | 0 |
| 247 | 426 | 100 | 98 | 98 | 0.98 | 98 | 1 | 1 | Flavonifractor | Parabacteroides | 0 |
| 247 | 586 | 100 | 98 | 98 | 0.98 | 98 | 1 | 1 | Flavonifractor | Subdoligranulum | 0 |
| 254 | 489 | 98 | 100 | 98 | 0.98 | 98 | 1 | 1 | Fusicatenibacter | Pseudobutyrivibrio | 0 |
| 254 | 526 | 98 | 100 | 98 | 0.98 | 98 | 1 | 1 | Fusicatenibacter | Roseburia | 0 |
| 302 | 489 | 98 | 100 | 98 | 0.98 | 98 | 1 | 1 | Intestinibacter | Pseudobutyrivibrio | 0 |
| 382 | 526 | 98 | 100 | 98 | 0.98 | 98 | 1 | 1 | Intestinibacter | Roseburia | 0 |
| 426 | 489 | 98 | 100 | 98 | 0.98 | 98 | 1 | 1 | Parabacteroides | Pseudobutyrivibrio | 0 |
| 426 | 526 | 98 | 100 | 98 | 0.98 | 98 | 1 | 1 | Parabacteroides | Roseburia | 0 |
| 489 | 586 | 100 | 98 | 98 | 0.98 | 98 | 1 | 1 | Pseudobutyrivibrio | Subdoligranulum | 0 |
| 526 | 586 | 100 | 98 | 98 | 0.98 | 98 | 1 | 1 | Roseburia | Subdoligranulum | 0 |
| 68 | 69 | 100 | 97 | 97 | 0.97 | 97 | 1 | 1 | Anaerostipes | Anaerotruncus | 0 |
| 68 | 325 | 100 | 97 | 97 | 0.97 | 97 | 1 | 1 | Anaerostipes | Lachnospira | 0 |
| 69 | 95 | 97 | 100 | 97 | 0.97 | 97 | 1 | 1 | Anaerotruncus | Bacteroides | 0 |
| 69 | 110 | 97 | 100 | 97 | 0.97 | 97 | 1 | 1 | Anaerotruncus | Blautia | 0 |
| 69 | 167 | 97 | 100 | 97 | 0.97 | 97 | 1 | 1 | Anaerotruncus | Clostridium | 0 |
| 69 | 213 | 97 | 100 | 97 | 0.97 | 97 | 1 | 1 | Anaerotruncus | Dorea | 0 |
| 69 | 237 | 97 | 100 | 97 | 0.97 | 97 | 1 | 1 | Anaerotruncus | Faecalibacterium | 0 |
| 69 | 247 | 97 | 100 | 97 | 0.97 | 97 | 1 | 1 | Anaerotruncus | Flavonifractor | 0 |
| 69 | 489 | 97 | 100 | 97 | 0.97 | 97 | 1 | 1 | Anaerotruncus | Pseudobutyrivibrio | 0 |
| 69 | 526 | 97 | 100 | 97 | 0.97 | 97 | 1 | 1 | Anaerotruncus | Roseburia | 0 |
| 95 | 325 | 100 | 97 | 97 | 0.97 | 97 | 1 | 1 | Bacteroides | Lachnospira | 0 |
| 110 | 325 | 100 | 97 | 97 | 0.97 | 97 | 1 | 1 | Blautia | Lachnospira | 0 |
| 167 | 325 | 100 | 97 | 97 | 0.97 | 97 | 1 | 1 | Clostridium | Lachnospira | 0 |
| 169 | 254 | 99 | 98 | 97 | 0.97 | 97 | 0.98 | 1 | Collinsella | Fusicatenibacter | 0 |
| 169 | 302 | 99 | 98 | 97 | 0.97 | 97 | 0.98 | 1 | Collinsella | Intestinibacter | 0 |
| 169 | 426 | 99 | 98 | 97 | 0.97 | 97 | 0.98 | 1 | Collinsella | Parabacteroides | 0 |
| 169 | 586 | 99 | 98 | 97 | 0.97 | 97 | 0.98 | 1 | Collinsella | Subdoligranulum | 0 |
| 213 | 325 | 100 | 97 | 97 | 0.97 | 97 | 1 | 1 | Dorea | Lachnospira | 0 |
| 231 | 254 | 99 | 98 | 97 | 0.97 | 97 | 0.98 | 1 | Erysipelatoclos- | Fusicatenibacter | 0 |
| | | | | | | | | | tridium | | |
| 231 | 302 | 99 | 98 | 97 | 0.97 | 97 | 0.98 | 1 | Erysipelatoclos- | Intestinibacter | 0 |
| | | | | | | | | | tridium | | |
| 231 | 426 | 99 | 98 | 97 | 0.97 | 97 | 0.98 | 1 | Erysipelatoclos- | Parabacteroides | 0 |
| | | | | | | | | | tridium | | |
| 231 | 586 | 99 | 98 | 97 | 0.97 | 97 | 0.98 | 1 | Erysipelatoclos- | Subdoligranulum | 0 |
| | | | | | | | | | tridium | | |
| 237 | 325 | 100 | 97 | 97 | 0.97 | 97 | 1 | 1 | Faecalibacterium | Lachnospira | 0 |
| 247 | 325 | 100 | 97 | 97 | 0.97 | 97 | 1 | 1 | Flavonifractor | Lachnospira | 0 |
| 284 | 539 | 98 | 99 | 97 | 0.97 | 97 | 0.98 | 1 | Fusicatenibacter | Sarcina | 0 |
| 302 | 539 | 98 | 99 | 97 | 0.97 | 97 | 0.98 | 1 | Intestinibacter | Sarcina | 0 |
| 325 | 489 | 97 | 100 | 97 | 0.97 | 97 | 1 | 1 | Lachnospira | Pseudobutyrivibrio | 0 |
| 325 | 526 | 97 | 100 | 97 | 0.97 | 97 | 1 | 1 | Lachnospira | Roseburia | 0 |
| 426 | 539 | 98 | 99 | 97 | 0.97 | 97 | 0.98 | 1 | Parabacteroides | Sarcina | 0 |
| 539 | 586 | 99 | 98 | 97 | 0.97 | 97 | 0.98 | 1 | Sarcina | Subdoligranulum | 0 |
| 69 | 169 | 97 | 99 | 96 | 0.96 | 96 | 0.97 | 1 | Anaerotruncus | Collinsella | 0 |
| 69 | 231 | 97 | 99 | 96 | 0.96 | 96 | 0.97 | 1 | Anaerotruncus | Erysipelatoclos- | 0 |
| | | | | | | | | | | tridium | |
| 69 | 539 | 97 | 99 | 96 | 0.96 | 96 | 0.97 | 1 | Anaerotruncus | Sarcina | 0 |
| 169 | 325 | 99 | 97 | 96 | 0.96 | 96 | 0.97 | 1 | Collinsella | Lachnospira | 0 |
| 231 | 325 | 99 | 97 | 96 | 0.96 | 96 | 0.97 | 1 | Erysipelatoclos- | Lachnospira | 0 |
| | | | | | | | | | tridium | | |
| 254 | 302 | 98 | 98 | 96 | 0.96 | 96 | 0.9602 | 1 | Fusicatenibacter | Intestinibacter | 0 |
| 254 | 426 | 98 | 98 | 96 | 0.96 | 96 | 0.9602 | 1 | Fusicatenibacter | Parabacteroides | 0 |
| 254 | 586 | 98 | 98 | 96 | 0.96 | 96 | 0.9602 | 1 | Fusicatenibacter | Subdoligranulum | 0 |
| 302 | 426 | 98 | 98 | 96 | 0.96 | 96 | 0.9602 | 1 | Intestinibacter | Parabacteroides | 0 |
| 302 | 586 | 98 | 98 | 96 | 0.96 | 96 | 0.9602 | 1 | Intestinibacter | Subdoligranulum | 0 |
| 325 | 539 | 97 | 99 | 97 | 0.96 | 96 | 1 | 0.03 | Lachnospira | Sarcina | 0.01 |
| 426 | 586 | 98 | 98 | 97 | 0.96 | 96 | 0.9998 | 0.0398 | Parabacteroides | Subdoligranulum | 0.01 |
| 69 | 254 | 97 | 98 | 96 | 0.951 | 95.1 | 0.99939 | 0.05939 | Anaerotruncus | Fusicatenibacter | 0.009 |
| 69 | 302 | 97 | 98 | 96 | 0.951 | 95.1 | 0.99939 | 0.05939 | Anaerotruncus | Intestinibacter | 0.009 |
| 69 | 426 | 97 | 98 | 95 | 0.951 | 95.1 | 0.94061 | 1 | Anaerotruncus | Parabacteroides | −0.001 |
| 69 | 586 | 97 | 98 | 95 | 0.951 | 95.1 | 0.94061 | 1 | Anaerotruncus | Subdoligranulum | −0.001 |
| 254 | 325 | 98 | 97 | 96 | 0.951 | 95.1 | 0.99939 | 0.05939 | Fusicatenibacter | Lachnospira | 0.009 |
| 302 | 325 | 98 | 97 | 95 | 0.951 | 95.1 | 0.94061 | 1 | Intestinibacter | Lachnospira | −0.001 |
| 325 | 426 | 97 | 98 | 95 | 0.951 | 95.1 | 0.94061 | 1 | Lachnospira | Parabacteroides | −0.001 |
| 325 | 586 | 97 | 98 | 95 | 0.951 | 95.1 | 0.94061 | 1 | Lachnospira | Subdoligranulum | −0.001 |
| 42 | 68 | 95 | 100 | 95 | 0.95 | 95 | 1 | 1 | Alistipes | Anaerostipes | 0 |
| 42 | 95 | 95 | 100 | 95 | 0.95 | 95 | 1 | 1 | Alistipes | Bacteroides | 0 |
| 42 | 110 | 95 | 100 | 95 | 0.95 | 95 | 1 | 1 | Alistipes | Blautia | 0 |
| 42 | 167 | 95 | 100 | 95 | 0.95 | 95 | 1 | 1 | Alistipes | Clostridium | 0 |
| 42 | 213 | 95 | 100 | 95 | 0.95 | 95 | 1 | 1 | Alistipes | Dorea | 0 |
| 42 | 237 | 95 | 100 | 95 | 0.95 | 95 | 1 | 1 | Alistipes | Faecalibacterium | 0 |
| 42 | 247 | 95 | 100 | 95 | 0.95 | 95 | 1 | 1 | Alistipes | Flavonifractor | 0 |
| 42 | 489 | 95 | 100 | 95 | 0.95 | 95 | 1 | 1 | Alistipes | Pseudobutyrivibrio | 0 |
| 42 | 526 | 95 | 100 | 95 | 0.95 | 95 | 1 | 1 | Alistipes | Roseburia | 0 |
| 68 | 303 | 100 | 95 | 95 | 0.95 | 95 | 1 | 1 | Anaerostipes | Intestinimonas | 0 |
| 95 | 303 | 100 | 95 | 95 | 0.95 | 95 | 1 | 1 | Bacteroides | Intestinimonas | 0 |
| 110 | 303 | 100 | 95 | 95 | 0.95 | 95 | 1 | 1 | Blautia | Intestinimonas | 0 |
| 167 | 303 | 100 | 95 | 95 | 0.95 | 95 | 1 | 1 | Clostridium | Intestinimonas | 0 |
| 213 | 303 | 100 | 95 | 95 | 0.95 | 95 | 1 | 1 | Dorea | Intestinimonas | 0 |
| 237 | 303 | 100 | 95 | 95 | 0.95 | 95 | 1 | 1 | Faecalibacterium | Intestinimonas | 0 |
| 247 | 303 | 100 | 95 | 95 | 0.95 | 95 | 1 | 1 | Flavonifractor | Intestinimonas | 0 |
| 303 | 489 | 95 | 100 | 95 | 0.95 | 95 | 1 | 1 | Intestinimonas | Pseudobutyrivibrio | 0 |
| 303 | 526 | 95 | 100 | 95 | 0.95 | 95 | 1 | 1 | Intestinimonas | Roseburia | 0 |
| 69 | 325 | 97 | 97 | 94 | 0.941 | 94.1 | 0.91181 | 1 | Anaerotruncus | Lachnospira | −0.001 |
| 42 | 169 | 95 | 99 | 94 | 0.94 | 94 | 0.95 | 1 | Alistipes | Collinsella | 0 |
| 42 | 231 | 95 | 99 | 94 | 0.94 | 94 | 0.95 | 1 | Alistipes | Erysipelatoclos- | 0 |
| | | | | | | | | | | tridium | |
| 42 | 539 | 95 | 99 | 95 | 0.94 | 94 | 1 | 0.05 | Alistipes | Sarcina | 0.01 |
| 169 | 303 | 99 | 95 | 95 | 0.94 | 94 | 1 | 0.05 | Collinsella | Intestinimonas | 0.01 |
| 231 | 303 | 99 | 95 | 94 | 0.94 | 94 | 0.95 | 1 | Erysipelatoclos- | Intestinimonas | 0 |
| | | | | | | | | | tridium | | |
| 303 | 539 | 95 | 99 | 94 | 0.94 | 94 | 0.95 | 1 | Intestinimonas | Sarcina | 0 |
| 42 | 254 | 95 | 98 | 94 | 0.931 | 93.1 | 0.99798 | 0.09798 | Alistipes | Fusicatenibacter | 0.009 |
| 42 | 302 | 95 | 98 | 93 | 0.931 | 93.1 | 0.90202 | 1 | Alistipes | Intestinibacter | −0.001 |
| 42 | 426 | 95 | 98 | 94 | 0.931 | 93.1 | 0.99798 | 0.09798 | Alistipes | Parabacteroides | 0.009 |
| 42 | 586 | 95 | 98 | 95 | 0.931 | 93.1 | 1 | 0.00202 | Alistipes | Subdoligranulum | 0.019 |
| 254 | 303 | 98 | 95 | 93 | 0.931 | 93.1 | 0.90202 | 1 | Fusicatenibacter | Intestinimonas | −0.001 |
| 302 | 303 | 98 | 95 | 93 | 0.931 | 93.1 | 0.90202 | 1 | Intestinibacter | Intestinimonas | −0.001 |
| 303 | 426 | 95 | 98 | 94 | 0.931 | 93.1 | 0.99798 | 0.09798 | Intestinimonas | Parabacteroides | 0.009 |
| 303 | 586 | 95 | 98 | 94 | 0.931 | 93.1 | 0.99798 | 0.09798 | Intestinimonas | Subdoligranulum | 0.009 |
| 68 | 584 | 100 | 93 | 93 | 0.93 | 93 | 1 | 1 | Anaerostipes | Streptococcus | 0 |
| 95 | 584 | 100 | 93 | 93 | 0.93 | 93 | 1 | 1 | Bacteroides | Streptococcus | 0 |
| 110 | 584 | 100 | 93 | 93 | 3.93 | 93 | 1 | 1 | Blautia | Streptococcus | 0 |
| 167 | 584 | 100 | 93 | 93 | 0.93 | 93 | 1 | 1 | Clostridium | Streptococcus | 0 |
| 213 | 584 | 100 | 93 | 93 | 0.93 | 93 | 1 | 1 | Dorea | Streptococcus | 0 |
| 237 | 584 | 100 | 93 | 93 | 0.93 | 93 | 1 | 1 | Faecalibacterium | Streptococcus | 0 |
| 247 | 584 | 100 | 93 | 93 | 0.93 | 93 | 1 | 1 | Flavonifractor | Streptococcus | 0 |
| 489 | 584 | 100 | 93 | 93 | 0.93 | 93 | 1 | 1 | Pseudobutyrivibrio | Streptococcus | 0 |
| 526 | 584 | 100 | 93 | 93 | 0.93 | 93 | 1 | 1 | Roseburia | Streptococcus | 0 |
| 42 | 69 | 95 | 97 | 93 | 0.922 | 92.2 | 0.99406 | 0.144 | Alistipes | Anaerotruncus | 0.008 |
| 42 | 325 | 95 | 97 | 93 | 0.922 | 92.2 | 0.99406 | 0.144 | Alistipes | Lachnospira | 0.008 |
| 69 | 303 | 97 | 95 | 92 | 0.922 | 92.2 | 0.856 | 1 | Anaerotruncus | Intestinimonas | −0.002 |
| 303 | 325 | 95 | 97 | 92 | 0.922 | 92.2 | 0.856 | 1 | Intestinimonas | Lachnospira | −0.002 |
| 169 | 584 | 99 | 93 | 93 | 0.921 | 92.1 | 1 | 0.07 | Collinsella | Streptococcus | 0.009 |
| 231 | 584 | 99 | 93 | 92 | 0.921 | 92.1 | 0.93 | 1 | Erysipelatoclos- | Streptococcus | −0.001 |
| | | | | | | | | | tridium | | |
| 539 | 584 | 99 | 93 | 92 | 0.921 | 92.1 | 0.93 | 1 | Sarcina | Streptococcus | −0.001 |
| 254 | 584 | 98 | 93 | 91 | 0.911 | 91.1 | 0.86424 | 1 | Fusicatenibacter | Streptococcus | −0.001 |
| 302 | 584 | 98 | 93 | 91 | 0.911 | 91.1 | 0.86424 | 1 | Intestinibacter | Streptococcus | −0.001 |
| 426 | 584 | 98 | 93 | 92 | 0.911 | 91.1 | 0.99576 | 0.13576 | Parabacteroides | Streptococcus | 0.009 |
| 584 | 586 | 93 | 98 | 91 | 0.911 | 91.1 | 0.86424 | 1 | Streptococcus | Subdoligranulum | −0.001 |
| 68 | 416 | 100 | 91 | 91 | 0.91 | 91 | 1 | 1 | Anaerostipes | Osciliibacter | 0 |
| 95 | 416 | 100 | 91 | 91 | 0.91 | 91 | 1 | 1 | Bacteroides | Oscillibacter | 0 |
| 110 | 416 | 100 | 91 | 91 | 0.91 | 91 | 1 | 1 | Blautia | Oscillibacter | 0 |
| 167 | 416 | 100 | 91 | 91 | 0.91 | 91 | 1 | 1 | Clostridium | Oscillibacter | 0 |
| 213 | 416 | 100 | 91 | 91 | 0.91 | 91 | 1 | 1 | Dorea | Oscillibacter | 0 |
| 237 | 416 | 100 | 91 | 91 | 0.91 | 91 | 1 | 1 | Faecalibacterium | Oscillibacter | 0 |
| 247 | 416 | 100 | 91 | 91 | 0.91 | 91 | 1 | 1 | Flavonifractor | Oscillibacter | 0 |
| 416 | 489 | 91 | 100 | 91 | 0.91 | 91 | 1 | 1 | Oscillibacter | Pseudobutyrivibrio | 0 |
| 416 | 526 | 91 | 100 | 91 | 0.91 | 91 | 1 | 1 | Oscillibacter | Roseburia | 0 |
| 42 | 303 | 95 | 95 | 91 | 0.902 | 90.2 | 0.98102 | 0.23041 | Alistipes | Intestinimonas | 0.008 |
| 69 | 584 | 97 | 93 | 90 | 0.902 | 90.2 | 0.80251 | 1 | Anaerotruncus | Streptococcus | −0.002 |
| 325 | 584 | 97 | 93 | 90 | 0.902 | 90.2 | 0.80251 | 1 | Lachnospira | Streptococcus | −0.002 |
| 169 | 416 | 99 | 91 | 91 | 0.901 | 90.1 | 1 | 0.09 | Collinsella | Oscillibacter | 0.009 |
| 231 | 416 | 99 | 91 | 90 | 0.901 | 90.1 | 0.91 | 1 | Erysipelatoclos- | Oscillibacter | −0.001 |
| | | | | | | | | | tridium | | |
| 416 | 539 | 91 | 99 | 90 | 0.901 | 90.1 | 0.91 | 1 | Oscillibacter | Sarcina | −0.001 |
| 254 | 416 | 98 | 91 | 89 | 0.892 | 89.2 | 0.82727 | 1 | Fusicatenibacter | Oscillibacter | −0.002 |
| 302 | 416 | 98 | 91 | 89 | 0.892 | 89.2 | 0.82727 | 1 | Intestinibacter | Oscillibacter | −0.002 |
| 416 | 426 | 91 | 98 | 89 | 0.892 | 89.2 | 0.82727 | 1 | Oscillibacter | Parabacteroides | −0.002 |
| 416 | 586 | 91 | 98 | 89 | 0.892 | 89.2 | 0.82727 | 1 | Oscillibacter | Subdoligranulum | −0.002 |
| 68 | 354 | 100 | 89 | 89 | 0.89 | 89 | 1 | 1 | Anaerostipes | Marvinbryantia | 0 |
| 95 | 354 | 100 | 89 | 89 | 0.89 | 89 | 1 | 1 | Bacteroides | Marvinbryantia | 0 |
| 110 | 354 | 100 | 89 | 89 | 0.89 | 89 | 1 | 1 | Blautia | Marvinbryantia | 0 |
| 167 | 354 | 100 | 89 | 89 | 0.89 | 89 | 1 | 1 | Clostridium | Marvinbryantia | 0 |
| 213 | 354 | 100 | 89 | 89 | 0.89 | 89 | 1 | 1 | Dorea | Marvinbryantia | 0 |
| 237 | 354 | 100 | 89 | 89 | 0.89 | 89 | 1 | 1 | Faecalibacterium | Marvinbryantia | 0 |
| 247 | 354 | 100 | 89 | 89 | 0.89 | 89 | 1 | 1 | Flavonifractor | Marvinbryantia | 0 |
| 354 | 489 | 89 | 100 | 89 | 0.89 | 89 | 1 | 1 | Marvinbryantia | Pseudobutyrivibrio | 0 |
| 354 | 526 | 89 | 100 | 89 | 0.89 | 89 | 1 | 1 | Marvinbryantia | Roseburia | 0 |
| 42 | 584 | 95 | 93 | 88 | 0.884 | 88.3 | 0.6903 | 1 | Alistipes | Streptococcus | −0.003 |
| 303 | 584 | 95 | 93 | 90 | 0.884 | 88.3 | 0.99797 | 0.03823 | Intestinimonas | Streptococcus | 0.017 |
| 69 | 416 | 97 | 91 | 88 | 0.883 | 88.3 | 0.7513 | 1 | Anaerotruncus | Oscillibacter | −0.003 |
| 325 | 416 | 97 | 91 | 88 | 0.883 | 88.3 | 0.7513 | 1 | Lachnospira | Oscillibacter | −0.003 |
| 169 | 354 | 99 | 89 | 88 | 0.881 | 88.1 | 0.89 | 1 | Collinsella | Marvinbryantia | −0.001 |
| 231 | 354 | 99 | 89 | 88 | 0.881 | 88.1 | 0.89 | 1 | Erysipelatoclos- | Marvinbryantia | −0.001 |
| | | | | | | | | | tridium | | |
| 354 | 539 | 89 | 99 | 89 | 0.881 | 88.1 | 1 | 0.11 | Marvinbryantia | Sarcina | 0.009 |
| 354 | 354 | 98 | 89 | 89 | 0.872 | 87.2 | 1 | 0.01111 | Fusicatenibacter | Marvinbryantia | 0.018 |
| 302 | 354 | 98 | 89 | 88 | 0.872 | 87.2 | 0.98889 | 0.20889 | Intestinibacter | Marvinbryantia | 0.008 |
| 354 | 426 | 89 | 98 | 88 | 0.872 | 87.2 | 0.98889 | 0.20889 | Marvinbryantia | Parabacteroides | 0.008 |
| 354 | 586 | 89 | 98 | 88 | 0.872 | 87.2 | 0.98889 | 0.20889 | Marvinbryantia | Subdoligranulum | 0.008 |
| 68 | 104 | 100 | 87 | 87 | 0.87 | 87 | 1 | 1 | Anaerostipes | Bilophila | 0 |
| 95 | 104 | 100 | 87 | 87 | 0.87 | 87 | 1 | 1 | Bacteroides | Bilophila | 0 |
| 104 | 110 | 87 | 100 | 87 | 0.87 | 87 | 1 | 1 | Bilophila | Blautia | 0 |
| 104 | 167 | 87 | 100 | 87 | 0.87 | 87 | 1 | 1 | Bilophila | Clostridium | 0 |
| 104 | 213 | 87 | 100 | 87 | 0.87 | 87 | 1 | 1 | Bilophila | Dorea | 0 |
| 104 | 237 | 87 | 100 | 87 | 0.87 | 87 | 1 | 1 | Bilophila | Faecalibacterium | 0 |
| 104 | 247 | 87 | 100 | 87 | 0.87 | 87 | 1 | 1 | Bilophila | Flavonifractor | 0 |
| 104 | 489 | 87 | 100 | 87 | 0.87 | 87 | 1 | 1 | Bilophila | Pseudobutyrivibrio | 0 |
| 104 | 526 | 87 | 100 | 87 | 0.87 | 87 | 1 | 1 | Bilophila | Roseburia | 0 |
| 42 | 416 | 95 | 91 | 86 | 0.864 | 86.4 | 0.61769 | 1 | Alistipes | Oscillibacter | −0.004 |
| 303 | 416 | 95 | 91 | 89 | 0.864 | 86.4 | 0.99985 | 0.00472 | Intestinimonas | Oscillibacter | 0.026 |
| 69 | 354 | 97 | 89 | 88 | 0.863 | 86.3 | 0.99898 | 0.03129 | Anaerotruncus | Marvinbryantia | 0.017 |
| 325 | 354 | 97 | 89 | 88 | 0.863 | 86.3 | 0.99898 | 0.03129 | Lachnospira | Marvinbryantia | 0.017 |
| 104 | 169 | 87 | 99 | 86 | 0.861 | 86.1 | 0.87 | 1 | Bilophila | Collinseiia | −0.001 |
| 104 | 231 | 87 | 99 | 86 | 0.861 | 86.1 | 0.87 | 1 | Bilophila | Erysipelatoclos- | −0.001 |
| | | | | | | | | | | tridium | |
| 104 | 539 | 87 | 99 | 86 | 0.861 | 86.1 | 0.87 | 1 | Bilophila | Sarcina | 0.007 |
| 104 | 254 | 87 | 98 | 86 | 0.853 | 85.3 | 0.98424 | 0.24424 | Bilophila | Fusicatenibacter | 0.007 |
| 104 | 302 | 87 | 98 | 86 | 0.853 | 85.3 | 0.98424 | 0.24424 | Bilophila | Intestinibacter | 0.001 |
| 104 | 426 | 87 | 98 | 85 | 0.853 | 85.3 | 0.75576 | 1 | Bitophila | Parabacteroides | −0.003 |
| 104 | 586 | 87 | 98 | 85 | 0.853 | 85.3 | 0.75576 | 1 | Bitophila | Subdoligranulum | −0.003 |

Using simple statistical tools, we could detect which bacteria are significantly different in antibiotic consumers. This list of taxa was later matched to a list of taxa having desired functional features to be important to recover after a course of antibiotics.

### Example 3: Method to Identify Bacteria Species being Applicable as Live Biotherapeutics

In the following section, specific examples of described potential bacteria to be used in a LBP (and/or suitable consumables (e.g., live biotherapeutics, probiotics, prebiotics, etc.)) are included.

According to our search, the consumption of antibiotics correlates with a decrease in butyrate and propionate-producing bacteria, as well as bacteria involved in the efficient digestion of polysaccharides. Specially, Bifidobacterium is a genus which has been shown not easy to recover after antibiotics consumption. Then, include those bacteria in a LBP formulation will help patients to recover of severe diarrhea and other detrimental effects after antibiotics usage.

Community services provided by the bacterial community in the gut are diverse, and usually redundant, meaning that more than only one taxon is involved in carrying out a certain function. As the microbiota decreases its abundance after antibiotic treatment, some of these functions are decreased or even disappear. In particular, we are interested in protecting and restoring the ability of the gut microbiota to produce short-chain fatty acids (SCFAs), which provide several benefits to humans. Other functions of relevance those are lost after antibiotics consumption can include one or more of: pathogen inhibition, degradation of polysaccharides, degradation of mucin, short-chain fatty acids production, production of conjugated linoleic acid, production of enterolactone, production of GABA, production of indole, and/or suitable microorganism-related functionality. In a specific example, thus, from the list of taxa that significantly change under antibiotic treatment, we looked for those that are involved in providing these functions of interest.

The inventors used the Metabolic Predictor tool developed by the Drug Development team and the previous literature search to identify the bacteria involved in the production of those molecules. Once the inventors knew which organisms had properties of producing butyrate and propionate, the inventors matched these organisms list with the list of taxa identified by logistic regression on all taxa that showed a decrease in their abundances in response to antibiotic consumption from Explorer Database (http://www.jenniebowers.com/explorer), to obtain the term of coeff_model_log in table 4 for the secondary selection of taxa. Then, based on the coeff_model_log and the functional features, the selected taxa are shown in Table 4.

The Explore allows users to easily obtain necessary information from the microbial taxa database, and similar microbial taxa databases can be used for this analysis and model construction. In addition, the microbial taxa database can be used continuously for increasing the accuracy of the analysis results as taxa data accumulates, and for analysis based on differences between specific groups (country, race, gender, aged, etc.).

There are a number of considerations about these analyzes. When using OTUs, the present inventors were using SILVA annotation to allocate that OTU to a specific taxon. The advantage of using SILVA annotation is that if a sequence cannot be allocated to one taxon, it moves up in the phylogeny and is annotated as Genus, Family, Class or so, making it more accurate, but we end up with fewer taxa annotated at the strain or species level.

In a first group, a new LBP formulation as an antibiotics recovery treatment can include any one or more strains (at any suitable amount) of the following species: Enterococcus faecium, Lactobacillus rhamnosus, Lactobacillus salivarius, Bifidobacterium adolescentis, Bifidobacterium animalis, Lactobacillus gasseri, Bifidobacterium breve, Bifidobacterium pseudocatenulatum, Lactobacillus reuteri, Lactobacillus fermentum, Pediococcus pentosaceus, Lactobacillus helveticus, Lactobacillus brevis, Lactococcus lactis. The combination of all of them, or a subset of them, can be used for treatment, diagnostics, and/or any suitable purpose. One or more of the described can include and/or be associated with all, or some of the following properties: pathogen inhibition, degradation of polysaccharides, degradation of mucin, short-chain fatty acids production, production of conjugated linoleic acid, production of enterolactone, production of GABA, production of indole, and/or suitable microorganism-related functionality.

In a second group, a new LBP formulation as an antibiotics recovery treatment can include any one or more strains (at any suitable amount) of the following species: Faecalibacterium prausnitzii, Roseburia faecis, Roseburia hominis, Roseburia intestinalis, Anaerostipes caccae, Anaerostipes rhamnosivorans, Eubacterium limosum, Eubacterium sp. ARC.2, Subdoligranulum variabile, Akkermansia muciniphila, Bifidobacterium adolescentis, Bifidobacterium animalis, Bifidobacterium breve, Bifidobacterium catenulatum, Bifidobacterium crudilactis, Bifidobacterium dentium, Bifidobacterium pseudocatenulatum, Bifidobacterium stercoris, Bifidobacterium thermacidophilum, Methanobrevibacter smithii, Roseburia sp. 499, Bacteroides dorei, Bacteroides massiliensis, Bacteroides plebeius, Bacteroides sp. 35AE37, Bacteroides thetaiotaomicron, Bacteroides xylanisolvens, Lactobacillus rhamnosus, Lactococcus lactis (table 4). The combination of all of them, or a subset of them, can be used for treatment, diagnostics, and/or any suitable purpose. One or more of the described can include and/or be associated with all, or some of the following properties: pathogenesis, pathogen inhibition, degradation of polysaccharides, degradation of mucin, short-chain fatty acids production, production of conjugated linoleic acid, production of enterolactone, production of GABA, production of indole, and/or suitable microorganism-related functionality.

In a specific example, the regression coefficient for each bacterial taxa, and some of their functions are described in the following list of Table 4. Bifidobacterium adolescentis, Bifidobacterium animalis, Bifidobacterium breve, Bifidobacterium catenulatum, Bifidobacterium pseudocatenulatum, and Bifidobacterium stercoris can be used in the probiotics.

TABLE 4
Potential bacteria to be used as LBP

| Bacterial Taxon | coeff_model_log | Functional feature |
|---|---|---|
| Faecalibacterium | −25.92 | polysaccharides-degrading |
| prausnitzii | | SCFA-producing |
| Roseburia faecis | −5.46 | polysaccharides-degrading |
| | | SCFA-producing |
| | | mucin-degrading |
| Roseburia hominis | −5.19 | polysaccharides-degrading |
| | | SCFA-producing |
| Roseburia intestinalis | −3.57 | polysaccharides-degrading |
| | | SCFA-producing |
| Anaerostipes caccae | −0.98 | SCFA-producing |
| Anaerostipes | −0.88 | SCFA-producing |
| rhamnosivorans | | |
| Eubacterium limosum | −0.4 | SCFA-producing |
| Eubacterium sp. ARC.2 | −0.4 | Enterolactone-producing |
| Subdoligranulum variabile | −0.4 | SCFA-producing |
| Akkermansia muciniphila | −0.17 | SCFA-producing |
| | | mucin-degrading |
| Bifidobacterium | −0.16 | SCFA-producing |
| adolescentis | | |
| Bifidobacterium animalis | −0.16 | Pathogen inhibition |
| Bifidobacterium breve | −0.15 | polysaccharides-degrading |
| Bifidobacterium | −0.15 | SCFA-producing |
| catenulatum | | |
| Bifidobacterium crudilactis | −0.14 | polysaccharides-degrading |
| Bifidobacterium dentium | −0.14 | GABA-producing |
| Bifidobacterium | −0.13 | SCFA-producing |
| pseudocatenulatur | | |
| Bifidobacterium stercoris | −0.1 | SCFA-producing |
| Bifidobacterium | −0.1 | ** |
| thermacidophilum | | |
| Methanobrevibacter | −0.1 | polysaccharides-degrading |
| smithii | | |
| Roseburia sp. 499 | −0.06 | polysaccharides-degrading |
| Bacteroides dorei | −0.05 | polysaccharides-degrading |
| | | Indole-producers |
| Bacteroides massiliensis | −0.05 | polysaccharides-degrading |
| | | Indole-producers |
| Bacteroides plebeius | −0.05 | polysaccharides-degrading |
| | | indole-producers |
| Bacteroides sp. 35AE37 | −0.03 | polysaccharides-degrading |
| | | Indole-producers |
| Bacteroides | −0.02 | polysaccharides-degrading |
| thetaiotaomicron | | SCFA-producing |
| | | Indole-producers |
| Bacteroides xylanisolvens | −0.02 | polysaccharides-degrading |
| | | SCFA-producing |
| | | Indole-producers |
| Lactobacillus rhamnosus | −0.24 | Pathogen inhibition |
| | | SCFA-producing |
| | | Conjugated linoleic |
| | | acid-producers |
| Lactococcus lactis | −0.01 | Pathogen inhibition |
| | | SCFA-producing |
| | | Conjugated linoleic |
| | | acid-producers |

One of the most important functions that are usually lost after antibiotics treatment, as described according our statistical analyses, is the production of short-chain fatty acids (SCFA), such as propionate or butyrate. This important function helps to prevent severe diarrhea after the antibiotics treatment, among other anti-Inflammatory features. The present inventors have identified several species that are currently included in probiotics that are decreased or depleted after antibiotics usage, and that we can include in a new blend of LBPs. Moreover, the present inventor identified some new SCFA-producer species that are not included in any probiotic, so we can patent their usage in a LBP.

1. A microbiota recovery composition for an antibiotic-inducing imbalance of gut microbiota, comprising at least a bacterium selected from the group consisting of Faecalibacterium prausnitzii, Roseburia faecis, Roseburia hominis, Roseburia intestinalis, Anaerostipes caccae, and Anaerostipes rhamnosivorans.

2. The microbiota recovery composition according to claim 1, wherein the microbiota recovery composition comprises at least a bacterium selected from the group consisting of Faecalibacterium prausnitzii, Roseburia faecis, Roseburia hominis, Roseburia intestinalis, Anaerostipes caccae, Anaerostipes rhamnosivorans, Eubacterium limosum, Subdoligranulum variabile, Lactobacillus rhamnosus, Akkermansia muciniphila, Bifidobacterium adolescentis, Bifidobacterium catenulatum, Bifidobacterium pseudocatenulatum, and Bifidobacterium stercoris.

3. The microbiota recovery composition according to claim 1, wherein the microbiota recovery composition comprises at least a bacterium selected from the group consisting of Faecalibacterium prausnitzii, Roseburia faecis, Roseburia hominis, Roseburia intestinalis, Anaerostipes caccae, Anaerostipes rhamnosivorans, Eubacterium limosum, Subdoligranulum variabile, Akkermansia muciniphila, and Bacteroides thetaiotaomicron

4. The microbiota recovery composition according to claim 1, wherein the microbiota recovery composition comprises at least a bacterium selected from the group consisting of Faecalibacterium prausnitzii, Roseburia faecis, Roseburia hominis, Roseburia intestinalis, Anaerostipes caccae, Anaerostipes rhamnosivorans, Eubacterium limosum, Subdoligranulum variabile, Lactobacillus rhamnosus, Akkermansia muciniphila, Bifidobacterium adolescentis, Bifidobacterium catenulatum, Bifidobacterium pseudocatenulatum, Bifidobacterium stercoris, Bacteroides thetaiotaomicron, Bacteroides xylanisolvens, and Lactococcus lactis.

5. The microbiota recovery composition according to claim 1, wherein the microbiota recovery composition comprises at least a bacterium selected from the group consisting of Faecalibacterium prausnitzii, Roseburia faecis, Roseburia hominis, Roseburia intestinalis, Anaerostipes caccae, Anaerostipes rhamnosivorans, Eubacterium limosum, Eubacterium sp. ARC.2, Subdoligranulum variabile, Akkermansia muciniphila, Bifidobacterium adolescentis, Bifidobacterium animalis, Bifidobacterium breve, Bifidobacterium catenulatum, Bifidobacterium crudilactis, Bifidobacterium dentium, Bifidobacterium pseudocatenulatum, Bifidobacterium stercoris, Bifidobacterium thermacidophilum, Methanobrevibacter smithii, Roseburia sp. 499, Bacteroides dorei, Bacteroides massiliensis, Bacteroides plebeius, Bacteroides sp. 35AE37, Bacteroides thetaiotaomicron, Bacteroides xylanisolvens, Lactobacillus rhamnosus, Lactococcus lactis.

6. The microbiota recovery composition according to claim 1, wherein the microbiota recovery composition further comprises at least a bacterium selected from the group consisting of Enterococcus faecium, Lactobacillus rhamnosus, Lactobacillus salivarius, Bifidobacterium adolescentis, Bifidobacterium animalis, Lactobacillus gasseri, Bifidobacterium breve, Bifidobacterium pseudocatenulatum, Lactobacillus reuteri, Lactobacillus fermentum, Pediococcus pentosaceus, Lactobacillus helveticus, Lactobacillus brevis, and Lactococcus lactis.

7. The microbiota recovery composition of claim 1, wherein the antibiotic-inducing imbalance comprises gastrointestinal disorder.

8. The microbiota recovery composition of claim 1, wherein the composition is a probiotic.

9. The microbiota recovery composition of claim 1, wherein the composition further comprises a prebiotic.

10. The microbiota recovery composition of claim 1, wherein the composition is formulated in unit dosage form as a solid, semi-solid, liquid, capsule, or powder.

11. A method of selecting a microorganism useful for recovering an antibiotic-inducing imbalance of gut microbiota in a subject, comprising:
(a) receiving an aggregate set of samples from a population of subjects,
(b) obtaining a relative abundance for each bacterial taxon in the population,
(c) selecting candidate bacteria by applying the relative abundances of the bacterial taxa from step (b) to a regression model and determining the correlation between the relative abundances of a first subset of the population of subjects consuming an antibiotic, and a second subset of the population of subjects not consuming the antibiotic, and
(d) selecting bacteria useful for a microbiota recovery composition for an antibiotic-inducing imbalance of gut microbiota, by applying functional features of bacteria to the selected candidate bacteria, to exclude bacteria having a harmful functional feature and to extract bacteria having a beneficial functional feature from the candidate bacteria and in step (c).

12. The method of claim 11, wherein the functional features is at least one selected from a group consisting of pathogenesis, pathogen inhibition, degradation of polysaccharides, degradation of mucin, short-chain fatty acids production, production of conjugated linoleic acid, production of enterolactone, production of GABA, and production of indole.

13. The method of claim 12, wherein the selecting candidate bacteria in the step (c) is performed by excluding pathogenic bacteria based on the functional feature of pathogenesis.

14. The method of claim 11, wherein the steps (a) and (b) are performed by (i) receiving an aggregate set of fecal samples obtained from a population of subjects; (ii) isolating nucleic acids from the fecal samples; (iii) amplifying nucleic acids with primers directed at a variable region of a bacterial 16S rRNA gene; and (iv) detecting nucleic acids associated with bacterial taxa in the population by 16S rRNA sequencing or hybridization array.

15. The method of claim 11, wherein the step (c) is performed by applying both analyses of a logistic regression model and a zero-inflated negative binomial regression model, and selecting candidate bacteria satisfying the statistical difference in relative abundances for the both analyses.

16. The method of claim 15, wherein the selected candidate bacteria of the first subset of the population is lower relative abundance than that of the second subset of the population.

17. The method of claim 11, wherein the selecting candidate bacteria in the step (c), further comprises analysis of the co-occurrence probability for the first subset of the population of subjects consuming an antibiotic, or the second subset of the population of subjects not consuming the antibiotic.

18. A method of ameliorating or treating an antibiotic-inducing imbalance of gut microbiota in a subject, comprising providing a microbiota recovery composition according to claim 1, to a subject with the antibiotic-inducing imbalance of gut microbiota.

19. The method of ameliorating or treating an antibiotic-inducing imbalance of gut microbiota in a subject, comprising:
(a) receiving an aggregate set of samples from a population of subjects,
(b) obtaining a relative abundance for each bacterial taxon in the population,
(c) selecting candidate bacteria by applying the relative abundances of the bacterial taxa from step (a) to a regression model to the correlation between the relative abundances of a first subset of the population of subjects consuming an antibiotic, and a second subset of the population of subjects not consuming the antibiotic,
(d) selecting bacteria used for a microbiota recovery composition for an antibiotic-inducing imbalance of gut microbiota, by applying the functional features to the selected candidate bacteria, to exclude bacteria having a harmful functional feature and to extract bacteria having a beneficial functional feature from the candidate bacteria in step (c), and
(e) providing a microbiota recovery composition to the subject with an antibiotic-inducing imbalance of gut microbiota.

20. A microbiota recovery composition according to claim 1, in the use of amelioration or treatment of an antibiotic-inducing imbalance of gut microbiota in a subject.
